(19) United States
(12) Patent Application Publication
(10) Pub. No.: US 2022/0169729 A1
(43) Pub. Date: Jun. 2, 2022

(54) METHOD FOR THE GENERATION OF A TRIVALENT ANTIBODY EXPRESSING CELL BY TARGETED INTEGRATION OF MULTIPLE EXPRESSION CASSETTES IN A DEFINED ORGANIZATION

(71) Applicant: Hoffmann-La Roche Inc., Little Falls, NJ (US)

(72) Inventors: Johannes Auer, Schwaigen (DE); Monika Fruehschuetz, Penzberg (DE); Ulrich Goepfert, Penzberg (DE); Christina-Lisa Hoeck, Bichl (DE)

(73) Assignee: Hoffmann-La Roche Inc., Little Falls, NJ (US)

(21) Appl. No.: 17/553,516

(22) Filed: Dec. 16, 2021

Related U.S. Application Data
(63) Continuation of application No. PCT/EP2020/066678, filed on Jun. 17, 2020.

(30) Foreign Application Priority Data
Jun. 19, 2019 (EP) 19181095.1

(51) Int. Cl. C07K 16/28; C07K 16/46

(52) U.S. Cl. CPC C07K 16/2809; C07K 16/468; C07K 2317/14; C07K 2317/64; C07K 2317/35; C07K 2317/526; C07K 2317/55; C07K 2317/31

(57) ABSTRACT

Herein is reported a method for producing a trivalent antibody comprising the steps of cultivating a mammalian cell comprising a deoxyribonucleic acid encoding the trivalent antibody, and recovering the trivalent antibody from the cell or the cultivation medium, wherein the deoxyribonucleic acid encoding the trivalent antibody is stably integrated into the genome of the mammalian cell and comprises in 5′- to 3′-direction a first expression cassette encoding the first heavy chain, a second expression cassette encoding the first light chain, a third expression cassette encoding the first light chain, a fourth expression cassette encoding the second heavy chain, a fifth expression cassette encoding the second light chain, and a sixth expression cassette encoding the second light chain, wherein the first heavy chain comprises from N- to C-terminus a first heavy chain variable domain, a CH1 domain, a first light chain variable domain, a CH1 domain, a hinge region, a CH2 domain and a CH3 domain, the second heavy chain comprises from N- to C-terminus the first heavy chain variable domain, a CH1 domain, a hinge region, a CH2 domain and a CH3 domain, the first light chain comprises from N- to C-terminus a second heavy chain variable domain and a CL domain, and the second light chain comprises from N- to C-terminus a second light chain variable domain and a CL domain, wherein the first heavy chain variable domain and the second light chain variable domain form a first binding site and the second heavy chain variable domain and the first light chain variable domain form a second binding site.

### CROSS-REFERENCE TO RELATED APPLICATIONS

This application is a continuation of International Application No. PCT/EP2020/066678 having an International filing date of Jun. 17, 2020, which claims benefit of priority to European Patent Application No. 19181095.1, filed Jun. 19, 2019, all of which are incorporated by reference in their entirety.

### SEQUENCE LISTING

This application contains a Sequence Listing which has been submitted electronically in ASCII format and is hereby incorporated by reference in its entirety. Said ASCII copy, created on Dec. 10, 2021, is named P35597-US_Sequence_Listing.txt and is 8,425 bytes in size.

### FIELD OF INVENTION

The current invention is in the field of cell line generation and polypeptide production. More precisely, herein is reported a recombinant mammalian cell, which has been obtained by a double recombinase mediated cassette exchange reaction, resulting in a specific expression cassette sequence being integrated into the genome of the mammalian cell. Said cell can be used in a method for the production of a trivalent antibody.

### BACKGROUND OF THE INVENTION

Secreted and glycosylated polypeptides, such as e.g. antibodies, are usually produced by recombinant expression in eukaryotic cells, either as stable or as transient expression.

One strategy for generating a recombinant cell expressing an exogenous polypeptide of interest involves the random integration of a nucleotide sequence encoding the polypeptide of interest followed by selection and isolation steps. This approach, however, has several disadvantages. First, functional integration of a nucleotide sequence into the genome of a cell as such is not only a rare event but, given the randomness as to where the nucleotide sequence integrates, these rare events result in a variety of gene expression and cell growth phenotypes. Such variation, known as “position effect variation”, originates, at least in part, from the complex gene regulatory networks present in eukaryotic cell genomes and the accessibility of certain genomic loci for integration and gene expression. Second, random integration strategies generally do not offer control over the number of nucleotide sequence copies integrated into the cell's genome. In fact, gene amplification methods are often used to achieve high-producing cells. Such gene amplification, however, can also lead to unwanted cell phenotypes, such as, e.g., with unstable cell growth and/or product expression. Third, because of the integration loci heterogeneity inherent in the random integration process, it is time-consuming and labor-intensive to screen thousands of cells after transfection to isolate those recombinant cells demonstrating a desirable level of expression of the polypeptide of interest. Even after isolating such cells, stable expression of the polypeptide of interest is not guaranteed and further screening may be required to obtain a stable commercial production cell. Fourth, polypeptides produced from cells obtained by random integration exhibit a high degree of sequence variance, which may be, in part, due to the mutagenicity of the selective agents used to select for a high level of polypeptide expression. Finally, the higher the complexity of the polypeptide to be produced, i.e. the higher the number of different polypeptides or polypeptide chains required to form the polypeptide of interest inside the cell, the more important gets the control of the expression ratio of the different polypeptides or polypeptide chains to each other. The control of the expression ratio is required to enable efficient expression, correct assembly and successful secretion in high expression yield of the polypeptide of interest.

Targeted integration by recombinase mediated cassette exchange (RMCE) is a method to direct foreign DNA specifically and efficiently to a pre-defined site in a eukaryotic host genome (Turan et al., J. Mol. Biol. 407 (2011) 193-221).

WO 2006/007850 discloses anti-rhesus D recombinant polyclonal antibody and methods of manufacture using site-specific integration into the genome of individual host cells.

Crawford, Y., et al. (Biotechnol. Prog. 29 (2013) 1307-1315) reported the fast identification of reliable hosts for targeted cell line development from a limited-genome screening using combined phiC31 integrase and CRE-Lox technologies.

WO 2013/006142 discloses a nearly homogenous population of genetically altered eukaryotic cells, having stably incorporated in its genome a donor cassette comprises a strong polyadenylation site operably linked to an isolated nucleic acid fragment comprising a targeting nucleic acid site and a selectable marker protein-coding sequence wherein the isolated nucleic acid fragment is flanked by a first recombination site and a second non-identical recombination site.

WO 2018/162517 discloses that depending i) on the expression cassette sequence and ii) on the distribution of the expression cassettes between the different expression vectors a high variation in expression yield and product quality was observed.

Tadauchi, T., et al. discloses utilizing a regulated targeted integration cell line development approach to systematically investigate what makes an antibody difficult to express (Biotechnol. Prog. 35 (2019) No. 2, 1-11).

WO 2016/079076 discloses T-cell activating bispecific antigen binding molecules against FolR1 and CD3. In Example 29 the generation of a bispecific FolR1/CD3-kappa-lambda antibody is described using transient transfection and a plasmid ratio of the three expression vectors was 1:1:1. In Example 36 the preparation of DP47 GS TCB is described by co-transfecting HEK293-EBNA cells with the corresponding expression vectors in a 1:2:1:1 ratio (“vector heavy chain Fc(hole)”:“vector light chain”:“vector light chain CrossFab”:“vector heavy chain Fc(knob)-FabCrossFab”). A similar disclosure is provided in WO 2017/055389 and WO 2016/020309.

WO 2014/033074 discloses a blood brain barrier shuttle. In Example 2 the transient production of a trivalent MAb31-scFab(8D3) is disclosed using three expression plasmids at equimolar plasmid ratio upon transfection.

WO 2017/184831 allegedly discloses site-specific integration and expression of recombinant proteins in eukaryotic cells, especially methods for improved expression of antibodies including bispecific antibodies in eukaryotic cells, particularly Chinese hamster (Cricetulus griseus) cell lines, by employing an expression-enhancing locus. The data in this document is presented in an anonymized way, thus, not allowing a conclusion what has actually been shown.

Rajendra, Y., et al. discloses that a single quad vector is a simple, yet effective, alternative approach for generation of stable CHO cell lines and may accelerate cell line generation for clinical hetero-mAb therapeutics (Biotechnol. Prog. 33 (2017) 469-477).

### SUMMARY OF THE INVENTION

Herein is reported a recombinant mammalian cell expressing a trivalent antibody, especially a bispecific, trivalent antibodies, such as a T-cell bispecific antibody (TCB). A trivalent antibody is a heteromultimeric polypeptide not naturally expressed by said mammalian cell. More specifically, a trivalent antibody is a heteromultimeric protein consisting of four polypeptides or polypeptide chains: one light chain, which is a full length light chain; a further light chain, which is a domain exchanged light chain; one heavy chain, which is a full length heavy chain; and a further heavy chain, which is an extended heavy chain comprising an addition domain exchanged heavy or light chain Fab fragment. To achieve expression of a trivalent antibody a recombinant nucleic acid comprising multiple different expression cassettes in a specific and defined sequence has been integrated into the genome of a mammalian cell.

Especially, the method according to the current invention can be used for the generation of T-cell bispecific antibodies (TCBs). These can have a format as described, e.g. in WO 2013/026831. Those molecules can simultaneously bind to CD3 (first specificity) on T-cells and to an antigen on a target (e.g. tumor) cell (second specificity) and thereby induce killing of target cells.

Herein is also reported a method for generating a recombinant mammalian cell expressing a trivalent antibody, especially a trivalent, bispecific antibody, more specifically a TCB and a method for producing a trivalent antibody, especially a trivalent, bispecific antibody, more specifically a TCB using said recombinant mammalian cell.

In one preferred embodiment the bispecific, trivalent antibody comprises
a) a first and a second Fab fragment that each specifically bind to a first antigen,
b) one domain exchanged Fab fragment that specifically binds to a second antigen in which the CH1 and the CL domain are exchanged for each other,
c) one Fc-region comprising a first heavy chain Fc-region polypeptide and a second heavy chain Fc-region polypeptide,
wherein the C-terminus of CH1 domain of the first Fab fragment is connected to the N-terminus of one of the heavy chain Fc-region polypeptides and the C-terminus of the CL-domain of the domain exchanged Fab fragment is connected to the N-terminus of the other heavy chain Fc-region polypeptide, and
wherein the C-terminus of the CH1 domain of the second Fab fragment is connected to the N-terminus of the VH domain of the first Fab fragment or to the N-terminus of the VH domain of the domain exchanged Fab fragment, and
wherein the first antigen or the second antigen is human CD3.

In one preferred embodiment the bispecific, trivalent antibody comprises
a) a first and a second Fab fragment that each specifically bind to a first antigen,
b) one domain exchanged Fab fragment that specifically binds to a second antigen in which the VH and the VL domain are exchanged for each other,
c) one Fc-region comprising a first heavy chain Fc-region polypeptide and a second heavy chain Fc-region polypeptide,
wherein the C-terminus of CH1 domain of the first Fab fragment is connected to the N-terminus of one of the heavy chain Fc-region polypeptides and the C-terminus of the CH1-domain of the domain exchanged Fab fragment is connected to the N-terminus of the other heavy chain Fc-region polypeptide, and
wherein the C-terminus of the CH1 domain of the second Fab fragment is connected to the N-terminus of the VH domain of the first Fab fragment or to the N-terminus of the VL domain of the domain exchanged Fab fragment, and
wherein the first antigen or the second antigen is human CD3.

In one preferred embodiment none of the first light chain and the second light chain of the trivalent, bispecific antibody is a common light chain or a universal light chain.

The current invention is based, at least in part, on the finding that the sequence of the different expression cassettes required for the expression of the heteromultimeric, trivalent antibody, i.e. the expression cassette organization, as integrated into the genome of a mammalian cell influences the expression yield of the trivalent antibody (e.g. of a TCB).

The current invention is based, at least in part, on the finding that by integrating a nucleic acid encoding the heteromultimeric, trivalent antibody (e.g. a TCB) that has a specific expression cassette organization into the genome of a mammalian cell efficient recombinant expression and production of the trivalent antibody (e.g. of a TCB) can be achieved.

It has been found that the defined expression cassette sequence can advantageously be integrated into the genome of a mammalian cell by a double recombinase mediated cassette exchange reaction.

One aspect according to the current invention is a method for producing a trivalent antibody (e.g. a TCB) comprising the steps of
a) cultivating a mammalian cell comprising a deoxyribonucleic acid encoding the trivalent antibody (e.g. a TCB) optionally under conditions suitable for the expression of the trivalent antibody (e.g. a TCB), and
b) recovering the trivalent antibody (e.g. a TCB) from the cell or the cultivation medium,
wherein the deoxyribonucleic acid encoding the trivalent antibody (e.g. a TCB) is stably integrated into the genome of the mammalian cell and comprises in 5′- to 3′-direction
either
1)
   a first expression cassette encoding the first heavy chain,
   a second expression cassette encoding the first light chain,
   a third expression cassette encoding the first light chain,
   a fourth expression cassette encoding the second heavy chain,
   a fifth expression cassette encoding the second light chain, and
   optionally a sixth expression cassette encoding the second light chain,
or
2)
   a first expression cassette encoding the first light chain,
   a second expression cassette encoding the first light chain,
   a third expression cassette encoding the first heavy chain,
   a fourth expression cassette encoding the second heavy chain,
   a fifth expression cassette encoding the second light chain, and
   a sixth expression cassette encoding the second light chain,
wherein the first to third expression cassettes are arranged unidirectional, the fourth to sixth expression cassettes are arranged unidirectional and in opposite direction as the first to third expression cassette;
or
3)
   a first expression cassette encoding the first heavy chain,
   a second expression cassette encoding the first light chain,
   a third expression cassette encoding the second light chain,
   a fourth expression cassette encoding the second heavy chain,
   a fifth expression cassette encoding the second light chain, and
   a sixth expression cassette encoding the first light chain,
or
   a first expression cassette encoding the first heavy chain,
   a second expression cassette encoding the first light chain,
   a third expression cassette encoding the second light chain,
   a fourth expression cassette encoding the second heavy chain,
   a fifth expression cassette encoding the second light chain, and
   a sixth expression cassette encoding the second light chain.

In one preferred embodiment the first heavy chain comprises in the CH3 domain the mutation T366W (numbering according to Kabat) and the second heavy chain comprises in the CH3 domain the mutations T366S, L368A, and Y407V, or vice versa (numbering according to Kabat). In one embodiment one of the heavy chains further comprises the mutation S354C and the respective other heavy chain comprises the mutation Y349C (numbering according to Kabat). In one embodiment the first heavy chain is an extended heavy chain comprising an additional domain exchanged Fab fragment. In one embodiment the first light chain is a domain exchanged light chain.

In one embodiment the deoxyribonucleic acid comprises a further expression cassette between the first and the second expression cassette encoding the second heavy chain.

In one embodiment
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment the deoxyribonucleic acid is stably integrated into the genome of the mammalian cell at a single site or locus.

One aspect of the current invention is a deoxyribonucleic acid encoding a trivalent antibody (e.g. a TCB) comprising in 5′- to 3′-direction
either
1)
   a first expression cassette encoding the first heavy chain,
   a second expression cassette encoding the first light chain,
   a third expression cassette encoding the first light chain,
   a fourth expression cassette encoding the second heavy chain,
   a fifth expression cassette encoding the second light chain, and
   optionally a sixth expression cassette encoding the second light chain,
or
2)
   a first expression cassette encoding the first light chain,
   a second expression cassette encoding the first light chain,
   a third expression cassette encoding the first heavy chain,
   a fourth expression cassette encoding the second heavy chain,
   a fifth expression cassette encoding the second light chain, and
   a sixth expression cassette encoding the second light chain,
wherein the first to third expression cassettes are arranged unidirectional, the fourth to sixth expression cassettes are arranged unidirectional and in opposite direction as the first to third expression cassette;
or
3)
   a first expression cassette encoding the first heavy chain,
   a second expression cassette encoding the first light chain,
   a third expression cassette encoding the second light chain,
   a fourth expression cassette encoding the second heavy chain,
   a fifth expression cassette encoding the second light chain, and
   a sixth expression cassette encoding the first light chain,
or
   a first expression cassette encoding the first heavy chain,
   a second expression cassette encoding the first light chain,
   a third expression cassette encoding the second light chain,
   a fourth expression cassette encoding the second heavy chain,
   a fifth expression cassette encoding the second light chain, and
   a sixth expression cassette encoding the second light chain.

In one preferred embodiment the first heavy chain comprises in the CH3 domain the mutation T366W (numbering according to Kabat) and the second heavy chain comprises in the CH3 domain the mutations T366S, L368A, and Y407V, or vice versa (numbering according to Kabat). In one embodiment one of the heavy chains further comprises the mutation S354C and the respective other heavy chain comprises the mutation Y349C (numbering according to Kabat). In one embodiment the first heavy chain is an extended heavy chain comprising an additional domain exchanged Fab fragment. In one embodiment the first light chain is a domain exchanged light chain.

In one embodiment the deoxyribonucleic acid comprises a further expression cassette between the first and the second expression cassette encoding the second heavy chain.

In one embodiment
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

One aspect of the current invention is the use of a deoxyribonucleic acid comprising in 5′- to 3′-direction
either
1)
   a first expression cassette encoding the first heavy chain,
   a second expression cassette encoding the first light chain,
   a third expression cassette encoding the first light chain,
   a fourth expression cassette encoding the second heavy chain,
   a fifth expression cassette encoding the second light chain, and
   optionally a sixth expression cassette encoding the second light chain,
or
2)
   a first expression cassette encoding the first light chain,
   a second expression cassette encoding the first light chain,
   a third expression cassette encoding the first heavy chain,
   a fourth expression cassette encoding the second heavy chain,
   a fifth expression cassette encoding the second light chain, and
   a sixth expression cassette encoding the second light chain,
wherein the first to third expression cassettes are arranged unidirectional, the fourth to sixth expression cassettes are arranged unidirectional and in opposite direction as the first to third expression cassette;
or
3)
   a first expression cassette encoding the first heavy chain,
   a second expression cassette encoding the first light chain,
   a third expression cassette encoding the second light chain,
   a fourth expression cassette encoding the second heavy chain,
   a fifth expression cassette encoding the second light chain, and
   a sixth expression cassette encoding the first light chain,
or
   a first expression cassette encoding the first heavy chain,
   a second expression cassette encoding the first light chain,
   a third expression cassette encoding the second light chain,
   a fourth expression cassette encoding the second heavy chain,
   a fifth expression cassette encoding the second light chain, and
   a sixth expression cassette encoding the second light chain,
for the expression of the trivalent antibody (e.g. a TCB) in a mammalian cell.

In one preferred embodiment the first heavy chain comprises in the CH3 domain the mutation T366W (numbering according to Kabat) and the second heavy chain comprises in the CH3 domain the mutations T366S, L368A, and Y407V, or vice versa (numbering according to Kabat). In one embodiment one of the heavy chains further comprises the mutation S354C and the respective other heavy chain comprises the mutation Y349C (numbering according to Kabat). In one embodiment the first heavy chain is an extended heavy chain comprising an additional domain exchanged Fab fragment. In one embodiment the first light chain is a domain exchanged light chain.

In one embodiment the deoxyribonucleic acid comprises a further expression cassette between the first and the second expression cassette encoding the second heavy chain.

In one embodiment
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment of the use the deoxyribonucleic acid is integrated into the genome of the mammalian cell.

In one embodiment of the use the deoxyribonucleic acid is stably integrated into the genome of the mammalian cell at a single site or locus.

One aspect of the invention is a recombinant mammalian cell comprising a deoxyribonucleic acid encoding a trivalent antibody (e.g. a TCB) integrated in the genome of the cell,
wherein the deoxyribonucleic acid encoding the trivalent antibody (e.g. a TCB) comprises in 5′- to 3′-direction
   either
   1)
      a first expression cassette encoding the first heavy chain,
      a second expression cassette encoding the first light chain,
      a third expression cassette encoding the first light chain,
      a fourth expression cassette encoding the second heavy chain,
      a fifth expression cassette encoding the second light chain, and
      optionally, a sixth expression cassette encoding the second light chain,
   or
   2)
      a first expression cassette encoding the first light chain,
      a second expression cassette encoding the first light chain,
      a third expression cassette encoding the first heavy chain,
      a fourth expression cassette encoding the second heavy chain,
      a fifth expression cassette encoding the second light chain, and
      a sixth expression cassette encoding the second light chain,
   wherein the first to third expression cassettes are arranged unidirectional, the fourth to sixth expression cassettes are arranged unidirectional and in opposite direction as the first to third expression cassette;
   or
   3)
      a first expression cassette encoding the first heavy chain,
      a second expression cassette encoding the first light chain,
      a third expression cassette encoding the second light chain,
      a fourth expression cassette encoding the second heavy chain,
      a fifth expression cassette encoding the second light chain, and
      a sixth expression cassette encoding the first light chain,
   or
      a first expression cassette encoding the first heavy chain,
      a second expression cassette encoding the first light chain,
      a third expression cassette encoding the second light chain,
      a fourth expression cassette encoding the second heavy chain,
      a fifth expression cassette encoding the second light chain, and
      a sixth expression cassette encoding the second light chain.

In one preferred embodiment the first heavy chain comprises in the CH3 domain the mutation T366W (numbering according to Kabat) and the second heavy chain comprises in the CH3 domain the mutations T366S, L368A, and Y407V, or vice versa (numbering according to Kabat). In one embodiment one of the heavy chains further comprises the mutation S354C and the respective other heavy chain comprises the mutation Y349C (numbering according to Kabat). In one embodiment the first heavy chain is an extended heavy chain comprising an additional domain exchanged Fab fragment. In one embodiment the first light chain is a domain exchanged light chain.

In one embodiment the deoxyribonucleic acid comprises a further expression cassette between the first and the second expression cassette encoding the second heavy chain.

In one embodiment of all previous aspects and embodiments
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment of all previous aspects and embodiments
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment the deoxyribonucleic acid is stably integrated into the genome of the mammalian cell at a single site or locus.

In one embodiment of all previous aspects and embodiments the deoxyribonucleic acid encoding the trivalent antibody (e.g. a TCB) further comprises
a first recombination recognition sequence located 5′ to the first (most 5′) expression cassette,
a second recombination recognition sequence located 3′ to the sixth (most 3′) expression cassette, and
a third recombination recognition sequence located
   between the first and the second recombination recognition sequence, and
   between two of the expression cassettes,
and
wherein all recombination recognition sequences are different.

In one embodiment of all previous aspects and embodiments the third recombination recognition sequence is located between the third and the fourth expression cassette.

In one embodiment of all previous aspects and embodiments the deoxyribonucleic acid encoding the trivalent antibody (e.g. a TCB) comprises a further expression cassette encoding for a selection marker and the expression cassette encoding for the selection marker is located partly 5′ and partly 3′ to the third recombination recognition sequence, wherein the 5′-located part of said expression cassette comprises the promoter and the start-codon and the 3′-located part of said expression cassette comprises the coding sequence without a start-codon and a polyA signal, wherein the start-codon is operably linked to the coding sequence.

One aspect of the current invention is a composition comprising two deoxyribonucleic acids, which comprise in turn three different recombination recognition sequences and six expression cassettes, wherein
the first deoxyribonucleic acid comprises in 5′- to 3′-direction,
   a first recombination recognition sequence,
   a first expression cassette encoding the first heavy chain,
   a second expression cassette encoding the first light chain,
   a third expression cassette encoding the first light chain, and
   a first copy of a third recombination recognition sequence,
and
the second deoxyribonucleic acid comprises in 5′- to 3′-direction
   a second copy of the third recombination recognition sequence,
   a fourth expression cassette encoding the second heavy chain,
   a fifth expression cassette encoding the second light chain,
   a sixth expression cassette encoding the second light chain, and
   a second recombination recognition sequence.

In one preferred embodiment the first heavy chain comprises in the CH3 domain the mutation T366W (numbering according to Kabat) and the second heavy chain comprises in the CH3 domain the mutations T366S, L368A, and Y407V, or vice versa (numbering according to Kabat). In one embodiment one of the heavy chains further comprises the mutation S354C and the respective other heavy chain comprises the mutation Y349C (numbering according to Kabat). In one embodiment the first heavy chain is an extended heavy chain comprising an additional domain exchanged Fab fragment. In one embodiment the first light chain is a domain exchanged light chain.

In one embodiment of all previous aspects and embodiments
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment of all previous aspects and embodiments
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment of all previous aspects and embodiments the deoxyribonucleic acid encoding the trivalent antibody (e.g. a TCB) further comprises a further expression cassette encoding for a selection marker.

In one embodiment of all previous aspects and embodiments the expression cassette encoding for a selection marker is located either
i) 5′, or
ii) 3′, or
iii) partly 5′ and partly 3′
   to the third recombination recognition sequence.

In one embodiment of all previous aspects and embodiments the expression cassette encoding for a selection marker is located partly 5′ and partly 3′ to the third recombination recognition sequences, wherein the 5′-located part of said expression cassette comprises the promoter and a start-codon and the 3′-located part of said expression cassette comprises the coding sequence without a start-codon and a polyA signal.

In one embodiment of all previous aspects and embodiments the 5′-located part of the expression cassette encoding the selection marker comprises a promoter sequence operably linked to a start-codon, whereby the promoter sequence is flanked upstream by (i.e. is positioned downstream to) the third expression cassette and the start-codon is flanked downstream by (i.e. is positioned upstream of) the third recombination recognition sequence; and the 3′-located part of the expression cassette encoding the selection marker comprises a nucleic acid encoding the selection marker lacking a start-codon and is flanked upstream by the third recombination recognition sequence and downstream by the fourth expression cassette.

In one embodiment of all previous aspects and embodiments the start-codon is a transcription start-codon. In one embodiment the start-codon is ATG.

One aspect of the invention is a recombinant mammalian cell comprising a deoxyribonucleic acid encoding a trivalent antibody (e.g. a TCB) integrated in the genome of the cell,
wherein the deoxyribonucleic acid encoding the trivalent antibody (e.g. a TCB) comprises the following elements:
a first, a second and a third recombination recognition sequence,
a first and a second selection marker, and
a first to sixth expression cassette,
   wherein the sequences of said elements in 5′-to-3′ direction is
      RRS1-1^{st} EC-2^{nd} EC-3^{rd} EC-RRS3-SM1-4^{th} EC-5^{th} EC-6^{th} EC-RRS2
   with
      RRS=recombination recognition sequence,
      EC=expression cassette,
      SM=selection marker.

One aspect of the current invention is a method for producing a recombinant mammalian cell comprising a deoxyribonucleic acid encoding a trivalent antibody (e.g. a TCB) and secreting the trivalent antibody (e.g. a TCB) comprising the following steps:
a) providing a mammalian cell comprising an exogenous nucleotide sequence integrated at a single site within a locus of the genome of the mammalian cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different;
b) introducing into the cell provided in a) a composition of two deoxyribonucleic acids comprising three different recombination recognition sequences and six expression cassettes, wherein
   the first deoxyribonucleic acid comprises in 5′- to 3′-direction,
      a first recombination recognition sequence,
      a first expression cassette encoding the first heavy chain,
      a second expression cassette encoding the first light chain,
      a third expression cassette encoding the first light chain,
      a 5′-terminal part of an expression cassette encoding one second selection marker, and
      a first copy of a third recombination recognition sequence,
   and
   the second deoxyribonucleic acid comprises in 5′- to 3′-direction
      a second copy of the third recombination recognition sequence,
      a 3′-terminal part of an expression cassette encoding the one second selection marker,
      a fourth expression cassette encoding the second heavy chain,
      a fifth expression cassette encoding the second light chain,
      a sixth expression cassette encoding the second light chain, and
      a second recombination recognition sequence,
wherein the first to third recombination recognition sequences of the first and second deoxyribonucleic acids are matching the first to third recombination recognition sequence on the integrated exogenous nucleotide sequence,
wherein the 5′-terminal part and the 3′-terminal part of the expression cassette encoding the one second selection marker when taken together form a functional expression cassette of the one second selection marker;
c) introducing
   i) either simultaneously with the first and second deoxyribonucleic acid of b); or
   ii) sequentially thereafter
   one or more recombinases,
   wherein the one or more recombinases recognize the recombination recognition sequences of the first and the second deoxyribonucleic acid; (and optionally wherein the one or more recombinases perform two recombinase mediated cassette exchanges;)
and
d) selecting for cells expressing the second selection marker and secreting the trivalent antibody (e.g. a TCB),
thereby producing a recombinant mammalian cell comprising a deoxyribonucleic acid encoding the trivalent antibody (e.g. a TCB) and secreting the trivalent antibody (e.g. a TCB).

In one preferred embodiment of all previous aspects and embodiments the first heavy chain comprises in the CH3 domain the mutation T366W (numbering according to Kabat) and the second heavy chain comprises in the CH3 domain the mutations T366S, L368A, and Y407V, or vice versa (numbering according to Kabat). In one embodiment one of the heavy chains further comprises the mutation S354C and the respective other heavy chain comprises the mutation Y349C (numbering according to Kabat). In one embodiment the first heavy chain is an extended heavy chain comprising an additional domain exchanged Fab fragment.

In one embodiment of all previous aspects and embodiments the first light chain is a domain exchanged light chain.

In one embodiment of all previous aspects and embodiments
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment of all previous aspects and embodiments
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one embodiment of all previous aspects and embodiments the expression cassette encoding the one second selection marker is located partly 5′ and partly 3′ to the third recombination recognition sequences, wherein the 5′-located part of said expression cassette comprises the promoter and the start-codon and said 3′-located part of the expression cassette comprises the coding sequence of the one second selection marker without a start-codon and a polyA signal.

In one embodiment of all previous aspects and embodiments the 5′-terminal part of the expression cassette encoding the one second selection marker comprises a promoter sequence operably linked to the start-codon, whereby the promoter sequence is flanked upstream by (i.e. is positioned downstream to) the expression cassettes and the start-codon is flanked downstream by (i.e. is positioned upstream of) the third recombination recognition sequence; and the 3′-terminal part of the expression cassette encoding the one second selection marker comprises the coding sequence of the one second selection marker lacking a start-codon flanked upstream by the third recombination recognition sequence and downstream by the expression cassettes.

In one embodiment of all previous aspects and embodiments the start-codon is a translation start-codon. In one embodiment the start-codon is ATG.

In one embodiment of all previous aspects and embodiments the first deoxyribonucleic acid is integrated into a first vector and the second deoxyribonucleic acid is integrated into a second vector.

In one embodiment of all previous aspects and embodiments each of the expression cassettes comprise in 5′-to-3′ direction a promoter, a coding sequence and a polyadenylation signal sequence optionally followed by a terminator sequence.

In one embodiment of all previous aspects and embodiments
i) the first expression cassette comprises in 5′-to-3′ direction a promoter, a nucleic acid encoding the first heavy chain, and a polyadenylation signal sequence and optionally a terminator sequence,
ii) the second expression cassette comprises in 5′-to-3′ direction a promoter, a nucleic acid encoding the first light chain, and a polyadenylation signal sequence and optionally a terminator sequence,
iii) the third expression cassette comprises in 5′-to-3′ direction a promoter, a nucleic acid encoding the first light chain, and a polyadenylation signal sequence and optionally a terminator sequence,
iv) the fourth expression cassette comprises in 5′-to-3′ direction a promoter, a nucleic acid encoding the second heavy chain, and a polyadenylation signal sequence and optionally a terminator sequence,
v) the fifth expression cassette comprises in 5′-to-3′ direction a promoter, a nucleic acid encoding the second light chain., and a polyadenylation signal sequence and optionally a terminator sequence,
vi) the sixth expression cassette comprises in 5′-to-3′ direction a promoter, a nucleic acid encoding the second light chain, and a polyadenylation signal sequence and optionally a terminator sequence, and
vii) the expression cassette encoding the selection marker comprises in 5′-to-3′ direction a promoter, a nucleic acid encoding the selection marker, and a polyadenylation signal sequence and optionally a terminator sequence.

In one embodiment of all previous aspects and embodiments the promoter is the human CMV promoter with or without intron A, the polyadenylation signal sequence is the bGH polyA site and the terminator is the hGT terminator.

A terminator sequence prevents the generation of very long RNA transcripts by RNA polymerase II, i.e. the read-through into the next expression cassette in the deoxyribonucleic acid according to the invention and used in the methods according to the invention. That is, the expression of one structural gene of interest is controlled by its own promoter.

Thus, by the combination of a polyadenylation signal and a terminator sequence efficient transcription termination is achieved. That is, read-through of the RNA polymerase II is prevented by the presence of double termination signals. The terminator sequence initiates complex resolution and promotes dissociation of RNA polymerase from the DNA template.

In one embodiment of all previous aspects and embodiments the promoter is the human CMV promoter with intron A, the polyadenylation signal sequence is the bGH polyadenylation signal sequence and the terminator is the hGT terminator except for the expression cassette of the selection marker, wherein the promoter is the SV40 promoter and the polyadenylation signal sequence is the SV40 polyadenylation signal sequence and a terminator is absent.

In one embodiment of all previous aspects and embodiments the mammalian cell is a CHO cell. In one embodiment the CHO cell is a CHO-K1 cell.

In one embodiment of all aspects and embodiments the trivalent antibody is a therapeutic antibody.

In one embodiment of all aspects and embodiments the trivalent, bispecific (therapeutic) antibody (TCB) comprises
a first and a second Fab fragment, wherein each binding site of the first and the second Fab fragment specifically bind to the second antigen,
a third Fab fragment, wherein the binding site of the third Fab fragment specifically binds to the first antigen, and wherein the third Fab fragment comprises a domain crossover such that the variable light chain domain (VL) and the variable heavy chain domain (VH) are replaced by each other, and
an Fc-region comprising a first Fc-region polypeptide and a second Fc-region polypeptide,
wherein the first and the second Fab fragment each comprise a heavy chain fragment and a full length light chain,
wherein the C-terminus of the heavy chain fragment of the first Fab fragment is fused to the N-terminus of the first Fc-region polypeptide,
wherein the C-terminus of the heavy chain fragment of the second Fab fragment is fused to the N-terminus of the variable light chain domain of the third Fab fragment and the C-terminus of the heavy chain constant domain 1 of the third Fab fragment is fused to the N-terminus of the second Fc-region polypeptide.

In one embodiment of all aspects and embodiments herein at least one selection marker expression cassette is oriented in the opposite direction as the antibody heavy and light chain expression cassettes.

In one embodiment of all aspects and embodiments herein the antibody heavy and light chain expression cassettes are arranged with respect to each other unidirectional (i.e. have the same orientation in 3′- to 5′-direction) and at least one of the selection marker expression cassette is arranged bidirectional with respect to the antibody heavy and light chain expression cassettes.

In one embodiment of all aspects and embodiments the trivalent antibody is an anti-CD3/CD20 bispecific antibody. In one embodiment the anti-CD3/CD20 bispecific antibody is a TCB with CD20 being the second antigen. In one embodiment the bispecific anti-CD3/CD20 antibody is RG6026. Such an antibody is reported in WO 2016/020309, which is incorporated herein by reference in its entirety.

In one embodiment of all aspects and embodiments the trivalent antibody is an anti-CD3/CEA bispecific antibody. In one embodiment the anti-CD3/CEA bispecific antibody is a TCB with CEA being the second antigen. In one embodiment the bispecific anti-CD3/CEA antibody is RO6958688 or RG7802 or cibisatamab. Such an antibody is reported in WO 2017/055389, which is incorporated herein by reference in its entirety.

In one embodiment of all previous aspects and embodiments none of the first light chain and the second light chain of the trivalent, bispecific antibody is a common light chain or a universal light chain.

In one embodiment of all previous aspects and embodiments the second heavy chain variable domain and the first light chain variable domain form a first binding site and the first heavy chain variable domain and the second light chain variable domain form a second binding site.

In one preferred embodiment of all aspects and embodiments the first binding site specifically binds to human CD3.

In one preferred embodiment of all aspects and embodiments the second binding site specifically binds to human CD3.

In one preferred embodiment of all aspects and embodiments exactly two deoxyribonucleic acids are comprised or introduced.

The individual expression cassettes in the deoxyribonucleic acid according to the invention are arranged sequentially. The distance between the end of one expression cassette and the start of the thereafter following expression cassette is only a few nucleotides, which were required for, i.e. result from, the cloning procedure.

In one embodiment of all previous aspects and embodiments two directly following expression cassettes are spaced at most 100 bps apart (i.e. from the end of the poly A signal sequence or the terminator sequence, respectively, until the start of the following promoter element are at most 100 basepairs (bps)). In one embodiment two directly following expression cassettes are spaced at most 50 bps apart. In one preferred embodiment two directly following expression cassettes are spaced at most 25 bps apart.

### DETAILED DESCRIPTION OF EMBODIMENTS OF THE INVENTION

The current invention is based, at least in part, on the finding that for the expression of a trivalent antibody (e.g. a TCB), which is a complex molecule comprising different polypeptides, i.e. which is a heteromultimer, the use of a defined and specific expression cassette organization results in efficient expression and production of the trivalent antibody (e.g. a TCB) in mammalian cells, such as CHO cells.

The current invention is based, at least in part, on the finding that double recombinase mediated cassette exchange (RMCE) can be used for producing a recombinant mammalian cell, such as a recombinant CHO cell, in which a defined and specific expression cassette sequence has been integrated into the genome, which in turn results in the efficient expression and production of a trivalent antibody (e.g. a TCB). The integration is effected at a specific site in the genome of the mammalian cell by targeted integration. Thereby it is possible to control the expression ratio of the different polypeptides of the heteromultimeric, trivalent antibody (e.g. a TCB) relative to each other. Thereby in turn an efficient expression, correct assembly and successful secretion in high expression yield of correctly folded and assembled trivalent antibody (e.g. a TCB) is achieved.

### I. Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In one embodiment the term about denotes a range of +/−10% of the thereafter following numerical value. In one embodiment the term about denotes a range of +/−5% of the thereafter following numerical value.

The term “comprising” also encompasses the term “consisting of”.

The term “CD20-TCB” as used herein denotes a CD20-targeting TCB (CD20-TCB; RG6026; anti-CD3/CD20 antibody in TCB format), which has a long half-life and high potency enabled by high-avidity bivalent binding to CD20 and head-to-tail orientation of B- and T-cell-binding domains in a previously characterized 2:1 TCB molecular format (see e.g. Bacac, M., et al., Clin. Cancer Res. 22 (2016) 3286-3297; Bacac, M., et al., Oncoimmunology 5 (2016) e1203498).

The term “mammalian cell comprising an exogenous nucleotide sequence” encompasses cells into which one or more exogenous nucleic acid(s) have been introduced, including the progeny of such cells and which are intended to form the starting point for further genetic modification. Thus, the term “a mammalian cell comprising an exogenous nucleotide sequence” encompasses a cell comprising an exogenous nucleotide sequence integrated at a single site within a locus of the genome of the mammalian cell, wherein the exogenous nucleotide sequence comprises at least a first and a second recombination recognition sequence (these recombinase recognition sequences are different) flanking at least one first selection marker. In one embodiment the mammalian cell comprising an exogenous nucleotide sequence is a cell comprising an exogenous nucleotide sequence integrated at a single site within a locus of the genome of the host cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different

The term “recombinant cell” as used herein denotes a cell after final genetic modification, such as, e.g., a cell expressing a polypeptide of interest and that can be used for the production of said polypeptide of interest at any scale. For example, “a mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”. Although the cell is still capable of performing further RMCE reactions, it is not intended to do so.

A “mammalian cell comprising an exogenous nucleotide sequence” and a “recombinant cell” are both “transformed cells”. This term includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated” composition is one which has been separated from a component of its natural environment. In some embodiments, a composition is purified to greater than 95% or 99% purity as determined by, for example, electrophoretic (e.g., SDS-PAGE, isoelectric focusing (IEF), capillary electrophoresis, CE-SDS) or chromatographic (e.g., size exclusion chromatography or ion exchange or reverse phase HPLC). For review of methods for assessment of e.g. antibody purity, see, e.g., Flatman, S. et al., J. Chrom. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from a component of its natural environment.

The term “integration site” denotes a nucleic acid sequence within a cell's genome into which an exogenous nucleotide sequence is inserted. In certain embodiments, an integration site is between two adjacent nucleotides in the cell's genome. In certain embodiments, an integration site includes a stretch of nucleotide sequences. In certain embodiments, the integration site is located within a specific locus of the genome of a mammalian cell. In certain embodiments, the integration site is within an endogenous gene of a mammalian cell.

The terms “vector” or “plasmid”, which can be used interchangeably, as used herein, refer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “binding to” denotes the binding of a binding site to its target, such as e.g. of an antibody binding site comprising an antibody heavy chain variable domain and an antibody light chain variable domain to the respective antigen. This binding can be determined using, for example, a BIAcore® assay (GE Healthcare, Uppsala, Sweden). That is, the term “binding (to an antigen)” denotes the binding of an antibody in an in vitro assay to its antigen(s). In one embodiment binding is determined in a binding assay in which the antibody is bound to a surface and binding of the antigen to the antibody is measured by Surface Plasmon Resonance (SPR). Binding means e.g. a binding affinity (K_{D}) of 10⁻⁸ M or less, in some embodiments of 10⁻¹³ to 10⁻⁸ M, in some embodiments of 10⁻¹³ to 10⁻⁹ M. The term “binding” also includes the term “specifically binding”.

For example, in one possible embodiment of the BIAcore® assay the antigen is bound to a surface and binding of the antibody, i.e. its binding site(s), is measured by surface plasmon resonance (SPR). The affinity of the binding is defined by the terms kₐ (association constant: rate constant for the association to form a complex), k_{d} (dissociation constant; rate constant for the dissociation of the complex), and K_{D} (k_{d}/kₐ). Alternatively, the binding signal of a SPR sensorgram can be compared directly to the response signal of a reference, with respect to the resonance signal height and the dissociation behaviors.

The term “binding site” denotes any proteinaceous entity that shows binding specificity to a target. This can be, e.g., a receptor, a receptor ligand, an anticalin, an affibody, an antibody, etc. Thus, the term “binding site” as used herein denotes a polypeptide that can specifically bind to or can be specifically bound by a second polypeptide.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located at a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′ end-independent manner.

As used herein, the term “flanking” refers to that a first nucleotide sequence is located at either a 5′- or 3′-end, or both ends of a second nucleotide sequence. The flanking nucleotide sequence can be adjacent to or at a defined distance from the second nucleotide sequence. There is no specific limit of the length of a flanking nucleotide sequence. For example, a flanking sequence can be a few base pairs or a few thousand base pairs.

Deoxyribonucleic acids comprise a coding and a non-coding strand. The terms “5′” and “3′” when used herein refer to the position on the coding strand.

As used herein, the term “exogenous” indicates that a nucleotide sequence does not originate from a specific cell and is introduced into said cell by DNA delivery methods, e.g., by transfection, electroporation, or transformation methods. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the “exogenous” sequence is introduced into the cell, e.g., via recombinant DNA technology.

Antibodies

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991).

The term “heavy chain” is used herein with its original meaning, i.e. denoting the two larger polypeptide chains of the four polypeptide chains forming an antibody (see, e.g., Edelman, G. M. and Gally J. A., J. Exp. Med. 116 (1962) 207-227). The term “larger” in this context can refer to any of molecular weight, length and amino acid number. The term “heavy chain” is independent from the sequence and number of individual antibody domains present therein. It is solely assigned based on the molecular weight of the respective polypeptide.

The term “light chain” is used herein with its original meaning, i.e. denoting the smaller polypeptide chains of the four polypeptide chains forming an antibody (see, e.g., Edelman, G. M. and Gally J. A., J. Exp. Med. 116 (1962) 207-227). The term “smaller” in this context can refer to any of molecular weight, length and amino acid number. The term “light chain” is independent from the sequence and number of individual antibody domains present therein. It is solely assigned based on the molecular weight of the respective polypeptide.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype, and the Kabat EU index numbering system (see pages 661-723) of Kabat, et al., Sequences of Proteins of Immunological Interest, 5th ed., Public Health Service, National Institutes of Health, Bethesda, Md. (1991) is used for the constant heavy chain domains (CH1, hinge, CH2 and CH3, which is herein further clarified by referring to “numbering according to Kabat EU index” in this case).

The term “antibody” herein is used in the broadest sense and encompasses various antibody structures, including but not limited to full length antibodies, monoclonal antibodies, multispecific antibodies (e.g., bispecific antibodies), and antibody-antibody fragment-fusions as well as combinations thereof.

The term “native antibody” denotes naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a heavy chain variable region (VH) followed by three heavy chain constant domains (CH1, CH2, and CH3), whereby between the first and the second heavy chain constant domain a hinge region is located. Similarly, from N- to C-terminus, each light chain has a light chain variable region (VL) followed by a light chain constant domain (CL). The light chain of an antibody may be assigned to one of two types, called kappa (κ) and lambda (λ), based on the amino acid sequence of its constant domain.

The term “full length antibody” denotes an antibody having a structure substantially similar to that of a native antibody. A full length antibody comprises two or more full length antibody light chains each comprising in N- to C-terminal direction a variable region and a constant domain, as well as two heavy chains each comprising in N- to C-terminal direction a variable region, a first constant domain, a hinge region, a second constant domain and a third constant domain. In contrast to a native antibody, a full length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full length antibody.

The term “antibody binding site” denotes a pair of a heavy chain variable domain and a light chain variable domain. To ensure proper binding to the antigen these variable domains are cognate variable domains, i.e. belong together. An antibody the binding site comprises at least three HVRs (e.g. in case of a VHH) or three-six HVRs (e.g. in case of a naturally occurring, i.e. conventional, antibody with a VH/VL pair). Generally, the amino acid residues of an antibody that are responsible for antigen binding are forming the binding site. These residues are normally contained in a pair of an antibody heavy chain variable domain and a corresponding antibody light chain variable domain. The antigen-binding site of an antibody comprises amino acid residues from the “hypervariable regions” or “HVRs”. “Framework” or “FR” regions are those variable domain regions other than the hypervariable region residues as herein defined. Therefore, the light and heavy chain variable domains of an antibody comprise from N- to C-terminus the regions FR1, HVR1, FR2, HVR2, FR3, HVR3 and FR4. Especially, the HVR3 region of the heavy chain variable domain is the region, which contributes most to antigen binding and defines the binding specificity of an antibody. A “functional binding site” is capable of specifically binding to its target. The term “specifically binding to” denotes the binding of a binding site to its target in an in vitro assay, in one embodiment in a binding assay.

Such binding assay can be any assay as long the binding event can be detected. For example, an assay in which the antibody is bound to a surface and binding of the antigen(s) to the antibody is measured by Surface Plasmon Resonance (SPR). Alternatively, a bridging ELISA can be used.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the heavy chain variable domain VH (H1, H2, H3), and three in the light chain variable domain VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, Md. (1991), NIH Publication 91-3242.);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ϑ, γ, and μ, respectively.

The term “heavy chain constant region” denotes the region of an immunoglobulin heavy chain that contains the constant domains, i.e. for a native immunoglobulin the CH1 domain, the hinge region, the CH2 domain and the CH3 domain or for a full length immunoglobulin the first constant domain, the hinge region, the second constant domain and the third constant domain. In one embodiment, a human IgG heavy chain constant region extends from Ala118 to the carboxyl-terminus of the heavy chain (numbering according to Kabat EU index). However, the C-terminal lysine (Lys447) of the constant region may or may not be present (numbering according to Kabat EU index). The term “constant region” denotes a dimer comprising two heavy chain constant regions, which can be covalently linked to each other via the hinge region cysteine residues forming inter-chain disulfide bonds.

The term “heavy chain Fc-region” denotes the C-terminal region of an immunoglobulin heavy chain that contains at least a part of the hinge region (middle and lower hinge region), the second constant domain, e.g. the CH2 domain, and the third constant domain, e.g. the CH3 domain. In one embodiment, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but in the C-terminal part identical thereto. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The constant region, more precisely the Fc-region, of an antibody (and the constant region likewise) is directly involved in complement activation, Clq binding, C3 activation and Fc receptor binding. While the influence of an antibody on the complement system is dependent on certain conditions, binding to Clq is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1 q binding and C3 activation, whereas IgG4 do not activate the complement system, do not bind C1q and do not activate C3. An “Fc-region of an antibody” is a term well known to the skilled artisan and defined on the basis of papain cleavage of antibodies.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci.

The term “valent” as used within the current application denotes the presence of a specified number of binding sites in an antibody. As such, the terms “bivalent”, “tetravalent”, and “hexavalent” denote the presence of two binding site, four binding sites, and six binding sites, respectively, in an antibody.

A “monospecific antibody” denotes an antibody that has a single binding specificity, i.e. specifically binds to one antigen. Monospecific antibodies can be prepared as full-length antibodies or antibody fragments (e.g. F(ab′)₂) or combinations thereof (e.g. full length antibody plus additional scFv or Fab fragments). A monospecific antibody does not need to be monovalent, i.e. a monospecific antibody may comprise more than one binding site specifically binding to the one antigen. A native antibody, for example, is monospecific but bivalent.

A “multispecific antibody” denotes an antibody that has binding specificities for at least two different epitopes on the same antigen or two different antigens. Multispecific antibodies can be prepared as full-length antibodies or antibody fragments (e.g. F(ab′)₂ bispecific antibodies) or combinations thereof (e.g. full length antibody plus additional scFv or Fab fragments). A multispecific antibody is at least bivalent, i.e. comprises two antigen binding sites. Also a multispecific antibody is at least bispecific. Thus, a bivalent, bispecific antibody is the simplest form of a multispecific antibody. Engineered antibodies with two, three or more (e.g. four) functional antigen binding sites have also been reported (see, e.g., US 2002/0004587 A1).

In certain embodiments, the antibody is a multispecific antibody, e.g. at least a bispecific antibody. Multispecific antibodies are monoclonal antibodies that have binding specificities for at least two different antigens or epitopes. In certain embodiments, one of the binding specificities is for a first antigen and the other is for a different second antigen. In certain embodiments, multispecific antibodies may bind to two different epitopes of the same antigen. Multispecific antibodies may also be used to localize cytotoxic agents to cells, which express the antigen.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553; using specific technology for making bispecific antibody fragments (see, e.g., Holliger, P., et al., Proc. Natl. Acad. Sci. USA 90 (1993) 6444-6448); and using single-chain Fv (scFv) dimers (see, e.g., Gruber, M., et al., J. Immunol. 152 (1994) 5368-5374); and preparing trispecific antibodies as described, e.g., in Tutt, A., et al., J. Immunol. 147 (1991) 60-69).

The antibody or fragment can also be a multispecific antibody as described in WO 2009/080251, WO 2009/080252, WO 2009/080253, WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520.

Bispecific antibodies are generally antibody molecules that specifically bind to two different, non-overlapping epitopes on the same antigen or to two epitopes on different antigens.

The term “non-overlapping” in this context indicates that an amino acid residue that is comprised within the first paratope of the bispecific Fab is not comprised in the second paratope, and an amino acid that is comprised within the second paratope of the bispecific Fab is not comprised in the first paratope.

The “knobs into holes” dimerization modules and their use in antibody engineering are described in Carter P.; Ridgway J. B. B.; Presta L. G.: Immunotechnology, Volume 2, Number 1, February 1996, pp. 73-73(1).

The CH3 domains in the heavy chains of an antibody can be altered by the “knob-into-holes” technology, which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerization of these two CH3 domains and thereby of the polypeptide comprising them. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

The mutation T366W in the CH3 domain (of an antibody heavy chain) is denoted as “knob-mutation” or “mutation knob” and the mutations T366S, L368A, Y407V in the CH3 domain (of an antibody heavy chain) are denoted as “hole-mutations” or “mutations hole” (numbering according to Kabat EU index). An additional inter-chain disulfide bridge between the CH3 domains can also be used (Merchant, A. M., et al., Nature Biotech. 16 (1998) 677-681) e.g. by introducing a S354C mutation into the CH3 domain of the heavy chain with the “knob-mutation” (denotes as “knob-cys-mutations” or “mutations knob-cys”) and by introducing a Y349C mutation into the CH3 domain of the heavy chain with the “hole-mutations” (denotes as “hole-cys-mutations” or “mutations hole-cys”) (numbering according to Kabat EU index).

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci USA 108 (2011) 11187-11192. Such antibodies are generally termed CrossMab.

Multispecific antibodies also comprise in one embodiment at least one Fab fragment including either a domain crossover of the CH1 and the CL domains as mentioned under item (i) above, or a domain crossover of the VH and the VL domains as mentioned under item (ii) above, or a domain crossover of the VH-CH1 and the VL-VL domains as mentioned under item (iii) above. In case of multispecific antibodies with domain crossover, the Fabs specifically binding to the same antigen(s) are constructed to be of the same domain sequence. Hence, in case more than one Fab with a domain crossover is contained in the multispecific antibody, said Fab(s) specifically bind to the same antigen.

A “humanized” antibody refers to an antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “recombinant antibody”, as used herein, denotes all antibodies (chimeric, humanized and human) that are prepared, expressed, created or isolated by recombinant means, such as recombinant cells. This includes antibodies isolated from recombinant cells such as NSO, HEK, BHK or CHO cells.

As used herein, the term “antibody fragment” refers to a molecule other than an intact antibody that comprises a portion of an intact antibody that binds the antigen to which the intact antibody binds, i.e. it is a functional fragment. Examples of antibody fragments include but are not limited to Fv; Fab; Fab′; Fab′-SH; F(ab′)2; bispecific Fab; diabodies; linear antibodies; single-chain antibody molecules (e.g., scFv or scFab).

### II. Compositions and Methods

Generally, for the recombinant large scale production of a polypeptide of interest, such as e.g. a therapeutic polypeptide, a cell stably expressing and secreting said polypeptide is required. This cell is termed “recombinant cell” or “recombinant production cell” and the process used for generating such a cell is termed “cell line development”. In the first step of the cell line development process a suitable host cell, such as e.g. a CHO cell, is transfected with a nucleic acid sequence suitable for expression of said polypeptide of interest. In a second step a cell stably expressing the polypeptide of interest is selected based on the co-expression of a selection marker, which had been co-transfected with the nucleic acid encoding the polypeptide of interest.

A nucleic acid encoding a polypeptide, i.e. the coding sequence, is called a structural gene. Such a structural gene is simple information and additional regulatory elements are required for expression thereof. Therefore, normally a structural gene is integrated in an expression cassette. The minimal regulatory elements needed for an expression cassette to be functional in a mammalian cell are a promoter functional in said mammalian cell, which is located upstream, i.e. 5′, to the structural gene, and a polyadenylation signal sequence functional in said mammalian cell, which is located downstream, i.e. 3′, to the structural gene. The promoter, the structural gene and the polyadenylation signal sequence are arranged in an operably linked form.

In case the polypeptide of interest is a heteromultimeric polypeptide that is composed of different (monomeric) polypeptides, not only a single expression cassette is required but a multitude of expression cassettes differing in the contained structural gene, i.e. at least one expression cassette for each of the different (monomeric) polypeptides of the heteromultimeric polypeptide. For example, a full length antibody is a heteromultimeric polypeptide comprising two copies of a light chain as well as two copies of a heavy chain. Thus, a full length antibody is composed of two different polypeptides. Therefore, two expression cassettes are required for the expression of a full length antibody, one for the light chain and one for the heavy chain. If, for example, the full length antibody is a bispecific antibody, i.e. the antibody comprises two different binding sites specifically binding to two different antigens, the light chains as well as the heavy chains are different from each other also. Thus, such a bispecific full length antibody is composed of four different polypeptides and four expression cassettes are required.

The expression cassette(s) for the polypeptide of interest is(are) in turn integrated into a so called “expression vector”. An “expression vector” is a nucleic acid providing all required elements for the amplification of said vector in bacterial cells as well as the expression of the comprised structural gene(s) in a mammalian cell. Typically, an expression vector comprises a prokaryotic plasmid propagation unit, e.g. for E. coli, comprising an origin of replication, and a prokaryotic selection marker, as well as a eukaryotic selection marker, and the expression cassettes required for the expression of the structural gene(s) of interest. An “expression vector” is a transport vehicle for the introduction of expression cassettes into a mammalian cell.

As outlined in the previous paragraphs, the more complex the polypeptide to be expressed is the higher also the number of required different expression cassettes is. Inherently with the number of expression cassettes also the size of the nucleic acid to be integrated into the genome of the host cell increases. Concomitantly also the size of the expression vector increases. But there is a practical upper limit to the size of a vector in the range of about 15 kbps above which handling and processing efficiency profoundly drops. This issue can be addressed by using two or more expression vectors. Thereby the expression cassettes can be split between different expression vectors each comprising only some of the expression cassettes.

Conventional cell line development (CLD) relies on the random integration (RI) of the vectors carrying the expression cassettes for the polypeptide of interest (SOI). In general, several vectors or fragments thereof integrate into the cell's genome if vectors are transfected by a random approach. Therefore, transfection processes based on RI are non-predictable.

Thus, by addressing the size problem with splitting expression cassettes between different expression vectors a new problem arises—the random number of integrated expression cassettes and the spatial distribution thereof.

Generally, the more expression cassettes for expression of a structural gene are integrated into the genome of a cell the higher the amount of the respective expressed polypeptide becomes. Beside the number of integrated expression cassettes also the site and the locus of the integration influences the expression yield. If, for example, an expression cassette is integrated at a site with low transcriptional activity in the cell's genome only a small amount of the encoded polypeptide is expressed. But, if the same expression cassette is integrated at a site in the cell's genome with high transcriptional activity a high amount of the encoded polypeptide is expressed.

This difference in expression is not causing problems as long as the expression cassettes for the different polypeptides of a heteromultimeric polypeptide are all integrated at the same frequency and at loci with comparable transcriptional activity. Under such circumstances all polypeptides of the multimeric polypeptide are expressed at the same amount and the multimeric polypeptide will be assembled correctly.

But this scenario is very unlikely and cannot be assured for molecules composed of more than two polypeptides. For example, in WO 2018/162517 it has been disclosed that depending i) on the expression cassette sequence and ii) on the distribution of the expression cassettes between the different expression vectors a high variation in expression yield and product quality was observed using RI. Without being bound by this theory, this observation is due to the fact that the different expression cassettes from the different expression vectors integrate with differing frequency and at different loci in the cell resulting in differential expression of the different polypeptides of the heteromultimeric polypeptide, i.e. at non-appropriate, different ratios. Thereby, some of the monomeric polypeptides are present at higher amount and others at a lower amount. This disproportion between the monomers of the heteromultimeric polypeptide causes non-complete assembly, mis-assembly as well as slow-down of the secretion rate. All of the before will result in lower expression yield of the correctly folded heteromultimeric polypeptide and a higher fraction of product-related by-products.

Unlike conventional RI CLD, targeted integration (TI) CLD introduces the transgene comprising the different expression cassettes at a predetermined “hot-spot” in the cell's genome. Also the introduction is with a defined ratio of the expression cassettes. Thereby, without being bound by this theory, all the different polypeptides of the heteromultimeric polypeptide are expressed at the same (or at least a comparable and only slightly differing) rate and at an appropriate ratio. Thereby the amount of correctly assembled heteromultimeric polypeptide should be increased and the fraction of product-related by-product should be reduced.

Also, given the defined copy number and the defined integration site, recombinant cells obtained by TI should have better stability compared to cells obtained by RI. Moreover, since the selection marker is only used for selecting cells with proper TI and not for selecting cells with a high level of transgene expression, a less mutagenic marker may be applied to minimize the chance of sequence variants (SVs), which is in part due to the mutagenicity of the selective agents like methotrexate (MTX) or methionine sulfoximine (MSX).

But it has now been found that the sequence of the expression cassettes, i.e. the expression cassette organization, in the transgene used in TI has a profound impact on trivalent antibody (e.g. TCB) expression.

The current invention uses a specific expression cassette organization with a defined number and sequence of the individual expression cassettes. This results in high expression yield and good product quality of the trivalent antibody (e.g. TCB) expressed in a mammalian cell.

For the defined integration of the transgene with the expression cassette sequence according to the current invention TI methodology is used. The current invention provides a novel method of generating a trivalent antibody (e.g. a TCB) expressing recombinant mammalian cells using a two-plasmid recombinase mediated cassette exchange (RMCE) reaction. The improvement lies, amongst other things, in the defined integration at the same locus in a defined sequence and thereby a high expression of the trivalent antibody (e.g. a TCB) and a reduced product-related by-product formation.

The presently disclosed subject matter not only provides methods for producing recombinant mammalian cells for stable large scale production of a trivalent antibody (e.g. a TCB) but also for recombinant mammalian cells that have high productivity of a trivalent antibody (e.g. a TCB) with advantageous by-product profile.

The two-plasmid RMCE strategy used herein allows for the insertion of multiple expression cassettes in the same TI locus.

II.a the Transgene and the Method According to the Invention

Herein is reported a recombinant mammalian cell expressing a trivalent antibody (e.g. a TCB). A trivalent antibody (e.g. a TCB) is a heteromultimeric polypeptide not naturally expressed by said mammalian cell. More specifically, a trivalent antibody (e.g. a TCB) is a heterodimeric protein consisting of four polypeptides: a first and a second light chain as well as a first and a second heavy chain. To achieve expression of a trivalent antibody (e.g. a TCB) a recombinant nucleic acid comprising multiple different expression cassettes in a specific and defined sequence has been integrated into the genome of a mammalian cell.

Herein is also reported a method for generating a recombinant mammalian cell expressing a trivalent antibody (e.g. a TCB) and a method for producing a trivalent antibody (e.g. a TCB) using said recombinant mammalian cell.

The current invention is based, at least in part, on the finding that the sequence of the different expression cassettes required for the expression of the heteromultimeric, trivalent antibody (e.g. TCB), i.e. the expression cassette organization, as integrated into the genome of a mammalian cell influences the expression yield of the trivalent antibody (e.g. a TCB).

The current invention is based, at least in part, on the finding that double recombinase mediated cassette exchange (RMCE) can be used for producing a recombinant mammalian cell, such as a recombinant CHO cell, in which a defined and specific expression cassette sequence has been integrated into the genome, which in turn results in the efficient expression and production of a trivalent antibody (e.g. a TCB). The integration is effected at a specific site in the genome of the mammalian cell by targeted integration. Thereby it is possible to control the expression ratio of the different polypeptides of the heteromultimeric polypeptide relative to each other. Thereby in turn an efficient expression, correct assembly and successful secretion in high expression yield of correctly folded and assembled trivalent antibody (e.g. TCB) is achieved.

As a trivalent antibody (e.g. a TCB) is a hetero-4-mer at least four different expression cassettes are required for the expression thereof: a first for the expression the first light chain, a second for the expression of a second light chain, the third for the expression of a first heavy chain and the fourth for the expression of a second heavy chain. Additionally, one or more further expression cassette(s) for positive selection marker(s) can be included.

In an example, to examine the effect of expression cassette organization on productivity in the TI host, RMCE pools were generated by transfecting two plasmids (front and back vector) containing different numbers and organizations of the individual chains of a trivalent antibody in the TCB format. After selection, recovery, and verification of RMCE by flow cytometry, the pools' productivity was evaluated in a 14-day fed batch production assay. For specific vector organizations an increase in titer compared to the reference pools was observed.

The effect of the antibody chain expression cassette organization on expression of five different TCBs was evaluated. TCB 1 to 5 all had a different targeting specificity. TCB 3 was tested with 4 different anti-CD3 binding sites.

For TCB-1 the following results have been obtained; the reference organizations are shaded in grey, k=heavy chain with knob mutation; h=heavy chain with hole mutations; l=light chain; xl=light chain with domain exchange:

The current invention is summarized below.

One independent aspect according to the current invention is a method for producing a trivalent antibody comprising the steps of
a) cultivating a mammalian cell comprising a deoxyribonucleic acid encoding the trivalent antibody, and
b) recovering the trivalent antibody from the cell or the cultivation medium,
wherein the deoxyribonucleic acid encoding the trivalent antibody is stably integrated into the genome of the mammalian cell and comprises in 5′- to 3′-direction
either (1)
   a first expression cassette encoding the first heavy chain,
   a second expression cassette encoding the first light chain,
   a third expression cassette encoding the first light chain,
   a fourth expression cassette encoding the second heavy chain, and
   a fifth expression cassette encoding the second light chain,
or (2)
   a first expression cassette encoding the first heavy chain,
   a second expression cassette encoding the first light chain,
   a third expression cassette encoding the first light chain,
   a fourth expression cassette encoding the second heavy chain,
   a fifth expression cassette encoding the second light chain,
   a sixth expression cassette encoding the second light chain,
or (3)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the second light chain,
or (4)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the first heavy chain,
   a sixth expression cassette encoding the second light chain,
or (5)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the second heavy chain,
   a sixth expression cassette encoding the second light chain,
   a seventh expression cassette encoding the second light chain,
or (6)
   a first expression cassette encoding the first light chain,
   a second expression cassette encoding the first light chain,
   a third expression cassette encoding the first heavy chain,
   a fourth expression cassette encoding the second heavy chain,
   a fifth expression cassette encoding the second light chain,
   a sixth expression cassette encoding the second light chain.

The stable integration of the deoxyribonucleic acid encoding the trivalent antibody into the genome of the mammalian cell can be done by any method known to a person of skill in the art as long as the specified sequence of expression cassettes is maintained.

One independent aspect according to the current invention is a deoxyribonucleic acid encoding a trivalent antibody comprising in 5′- to 3′-direction
either (1)
   a first expression cassette encoding the first heavy chain,
   a second expression cassette encoding the first light chain,
   a third expression cassette encoding the first light chain,
   a fourth expression cassette encoding the second heavy chain, and
   a fifth expression cassette encoding the second light chain,
or (2)
   a first expression cassette encoding the first heavy chain,
   a second expression cassette encoding the first light chain,
   a third expression cassette encoding the first light chain,
   a fourth expression cassette encoding the second heavy chain,
   a fifth expression cassette encoding the second light chain,
   a sixth expression cassette encoding the second light chain,
or (3)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the second light chain,
or (4)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the first heavy chain,
   a sixth expression cassette encoding the second light chain,
or (5)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the second heavy chain,
   a sixth expression cassette encoding the second light chain,
   a seventh expression cassette encoding the second light chain,
or (6)
   a first expression cassette encoding the first light chain,
   a second expression cassette encoding the first light chain,
   a third expression cassette encoding the first heavy chain,
   a fourth expression cassette encoding the second heavy chain,
   a fifth expression cassette encoding the second light chain,
   a sixth expression cassette encoding the second light chain.

One independent aspect according to the current invention is the use of a deoxyribonucleic acid comprising in 5′- to 3′-direction
either (1)
   a first expression cassette encoding the first heavy chain,
   a second expression cassette encoding the first light chain,
   a third expression cassette encoding the first light chain,
   a fourth expression cassette encoding the second heavy chain, and
   a fifth expression cassette encoding the second light chain,
or (2)
   a first expression cassette encoding the first heavy chain,
   a second expression cassette encoding the first light chain,
   a third expression cassette encoding the first light chain,
   a fourth expression cassette encoding the second heavy chain,
   a fifth expression cassette encoding the second light chain,
   a sixth expression cassette encoding the second light chain,
or (3)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the second light chain,
or (4)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the first heavy chain,
   a sixth expression cassette encoding the second light chain,
or (5)
   a first expression cassette encoding the first heavy chain,
   a second expression cassette encoding the second heavy chain,
   a third expression cassette encoding the first light chain,
   a fourth expression cassette encoding the second light chain,
   a fifth expression cassette encoding the second heavy chain,
   a sixth expression cassette encoding the second light chain,
   a seventh expression cassette encoding the second light chain,
or (6)
   a first expression cassette encoding the first light chain,
   a second expression cassette encoding the first light chain,
   a third expression cassette encoding the first heavy chain,
   a fourth expression cassette encoding the second heavy chain,
   a fifth expression cassette encoding the second light chain,
   a sixth expression cassette encoding the second light chain,
for the expression of the trivalent antibody in a mammalian cell.

One independent aspect according to the current invention is a recombinant mammalian cell comprising a deoxyribonucleic acid encoding a trivalent antibody integrated in the genome of the cell,

wherein the deoxyribonucleic acid encoding the trivalent antibody comprises in 5′- to 3′-direction
either (1)
a first expression cassette encoding the first heavy chain,
a second expression cassette encoding the first light chain,
a third expression cassette encoding the first light chain,
a fourth expression cassette encoding the second heavy chain, and
a fifth expression cassette encoding the second light chain,
or (2)
a first expression cassette encoding the first heavy chain,
a second expression cassette encoding the first light chain,
a third expression cassette encoding the first light chain,
a fourth expression cassette encoding the second heavy chain,
a fifth expression cassette encoding the second light chain,
a sixth expression cassette encoding the second light chain,
or (3)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second light chain,
or (4)
a first expression cassette encoding the first heavy chain,
a second expression cassette encoding the second heavy chain,
a third expression cassette encoding the first light chain,
a fourth expression cassette encoding the second light chain,
a fifth expression cassette encoding the first heavy chain,
a sixth expression cassette encoding the second light chain,
or (5)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second heavy chain,
a sixth expression cassette encoding the second light chain,
a seventh expression cassette encoding the second light chain,
or (6)
a first expression cassette encoding the first light chain,
a second expression cassette encoding the first light chain,
a third expression cassette encoding the first heavy chain,
a fourth expression cassette encoding the second heavy chain,
a fifth expression cassette encoding the second light chain,
a sixth expression cassette encoding the second light chain.

One independent aspect according to the current invention is a composition comprising two deoxyribonucleic acids, which comprise in turn three different recombination recognition sequences and five to seven expression cassettes, wherein
the first deoxyribonucleic acid comprises in 5′- to 3′-direction,
   either (1)
      a first recombination recognition sequence,
      a first expression cassette encoding the first heavy chain,
      a second expression cassette encoding the first light chain,
      a third expression cassette encoding the first light chain, and
      a first copy of a third recombination recognition sequence,
   or (2)
      a first recombination recognition sequence,
      a first expression cassette encoding the first heavy chain,
      a second expression cassette encoding the first light chain,
      a third expression cassette encoding the first light chain, and
      a first copy of a third recombination recognition sequence,
   or (3)
      a first recombination recognition sequence,
      a first expression cassette encoding the first heavy chain,
      a second expression cassette encoding the second heavy chain,
      a third expression cassette encoding the first light chain,
      a fourth expression cassette encoding the second light chain, and
      a first copy of a third recombination recognition sequence,
   or (4)
      a first recombination recognition sequence,
      a first expression cassette encoding the first heavy chain,
      a second expression cassette encoding the second heavy chain,
      a third expression cassette encoding the first light chain,
      a fourth expression cassette encoding the second light chain, and
      a first copy of a third recombination recognition sequence,
   or (5)
      a first recombination recognition sequence,
      a first expression cassette encoding the first heavy chain,
      a second expression cassette encoding the second heavy chain,
      a third expression cassette encoding the first light chain,
      a fourth expression cassette encoding the second light chain, and
      a first copy of a third recombination recognition sequence,
   or (6)
      a first recombination recognition sequence,
      a first expression cassette encoding the first light chain,
      a second expression cassette encoding the first light chain,
      a third expression cassette encoding the first heavy chain, and
      a first copy of a third recombination recognition sequence,
and
   the second deoxyribonucleic acid comprises in 5′- to 3′-direction
      either (1)
         a second copy of the third recombination recognition sequence,
         a fourth expression cassette encoding the second heavy chain,
         a fifth expression cassette encoding the second light chain, and
         a second recombination recognition sequence,
      or (2)
         a second copy of the third recombination recognition sequence,
         a fourth expression cassette encoding the second heavy chain,
         a fifth expression cassette encoding the second light chain,
         a sixth expression cassette encoding the second light chain, and
         a second recombination recognition sequence,
      or (3)
         a second copy of the third recombination recognition sequence,
         a fifth expression cassette encoding the second light chain, and
         a second recombination recognition sequence,
      or (4)
         a second copy of the third recombination recognition sequence,
         a fifth expression cassette encoding the first heavy chain,
         a sixth expression cassette encoding the second light chain, and
         a second recombination recognition sequence,
      or (5)
         a second copy of the third recombination recognition sequence,
         a fifth expression cassette encoding the second heavy chain,
         a sixth expression cassette encoding the second light chain,
         a seventh expression cassette encoding the second light chain, and
         a second recombination recognition sequence,
      or (6)
         a second copy of the third recombination recognition sequence,
         a fourth expression cassette encoding the second heavy chain,
         a fifth expression cassette encoding the second light chain,
         a sixth expression cassette encoding the second light chain, and
         a second recombination recognition sequence.

In one embodiment the first and the second deoxyribonucleic acid both comprises the organization according to (1); or the first and the second deoxyribonucleic acid both comprises the organization according to (2); or the first and the second deoxyribonucleic acid both comprises the organization according to (3); or the first and the second deoxyribonucleic acid both comprises the organization according to (4); or the first and the second deoxyribonucleic acid both comprises the organization according to (5); or the first and the second deoxyribonucleic acid both comprises the organization according to (6).

One independent aspect according to the current invention is a method for producing a recombinant mammalian cell comprising a deoxyribonucleic acid encoding a trivalent antibody and secreting the trivalent antibody, comprising the following steps:
a) providing a mammalian cell comprising an exogenous nucleotide sequence integrated at a single site within a locus of the genome of the mammalian cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different;
b) introducing into the cell provided in a) a composition of two deoxyribonucleic acids comprising three different recombination recognition sequences and five to seven expression cassettes, wherein
   the first deoxyribonucleic acid comprises in 5′- to 3′-direction,
      either (1)
         a first recombination recognition sequence,
         a first expression cassette encoding the first heavy chain,
         a second expression cassette encoding the first light chain,
         a third expression cassette encoding the first light chain, and
         a first copy of a third recombination recognition sequence,
      or (2)
         a first recombination recognition sequence,
         a first expression cassette encoding the first heavy chain,
         a second expression cassette encoding the first light chain,
         a third expression cassette encoding the first light chain, and
         a first copy of a third recombination recognition sequence,
      or (3)
         a first recombination recognition sequence,
         a first expression cassette encoding the first heavy chain,
         a second expression cassette encoding the second heavy chain,
         a third expression cassette encoding the first light chain,
         a fourth expression cassette encoding the second light chain, and
         a first copy of a third recombination recognition sequence,
      or (4)
         a first recombination recognition sequence,
         a first expression cassette encoding the first heavy chain,
         a second expression cassette encoding the second heavy chain,
         a third expression cassette encoding the first light chain,
         a fourth expression cassette encoding the second light chain, and
         a first copy of a third recombination recognition sequence,
      or (5)
         a first recombination recognition sequence,
         a first expression cassette encoding the first heavy chain,
         a second expression cassette encoding the second heavy chain,
         a third expression cassette encoding the first light chain,
         a fourth expression cassette encoding the second light chain, and
         a first copy of a third recombination recognition sequence,
      or (6)
         a first recombination recognition sequence,
         a first expression cassette encoding the first light chain,
         a second expression cassette encoding the first light chain,
         a third expression cassette encoding the first heavy chain, and
         a first copy of a third recombination recognition sequence,
      and
         the second deoxyribonucleic acid comprises in 5′- to 3′-direction
      either (1)
         a second copy of the third recombination recognition sequence,
         a fourth expression cassette encoding the second heavy chain,
         a fifth expression cassette encoding the second light chain, and
         a second recombination recognition sequence,
      or (2)
         a second copy of the third recombination recognition sequence,
         a fourth expression cassette encoding the second heavy chain,
         a fifth expression cassette encoding the second light chain,
         a sixth expression cassette encoding the second light chain, and
         a second recombination recognition sequence,
      or (3)
         a second copy of the third recombination recognition sequence,
         a fifth expression cassette encoding the second light chain, and
         a second recombination recognition sequence,
      or (4)
         a second copy of the third recombination recognition sequence,
         a fifth expression cassette encoding the first heavy chain,
         a sixth expression cassette encoding the second light chain, and
         a second recombination recognition sequence,
      or (5)
         a second copy of the third recombination recognition sequence,
         a fifth expression cassette encoding the second heavy chain,
         a sixth expression cassette encoding the second light chain,
         a seventh expression cassette encoding the second light chain, and
         a second recombination recognition sequence,
      or (6)
         a second copy of the third recombination recognition sequence,
         a fourth expression cassette encoding the second heavy chain,
         a fifth expression cassette encoding the second light chain,
         a sixth expression cassette encoding the second light chain, and
         a second recombination recognition sequence,
   wherein the first to third recombination recognition sequences of the first and second deoxyribonucleic acids are matching the first to third recombination recognition sequence on the integrated exogenous nucleotide sequence,
   wherein the 5′-terminal part and the 3′-terminal part of the expression cassette encoding the one second selection marker when taken together form a functional expression cassette of the one second selection marker;
c) introducing
   i) either simultaneously with the first and second deoxyribonucleic acid of b);
   or
   ii) sequentially thereafter
   one or more recombinases,
   wherein the one or more recombinases recognize the recombination recognition sequences of the first and the second deoxyribonucleic acid; (and optionally wherein the one or more recombinases perform two recombinase mediated cassette exchanges)
and
d) selecting for cells expressing the second selection marker and secreting the trivalent antibody,
thereby producing a recombinant mammalian cell comprising a deoxyribonucleic acid encoding the trivalent antibody and secreting the trivalent antibody.

In one embodiment the first and the second deoxyribonucleic acid both comprises the organization according to (1); or the first and the second deoxyribonucleic acid both comprises the organization according to (2); or the first and the second deoxyribonucleic acid both comprises the organization according to (3); or the first and the second deoxyribonucleic acid both comprises the organization according to (4); or the first and the second deoxyribonucleic acid both comprises the organization according to (5); or the first and the second deoxyribonucleic acid both comprises the organization according to (6).

In one embodiment of all independent aspects as well as of all dependent embodiments of the current invention the deoxyribonucleic acid encoding the trivalent, bispecific antibody is stably integrated into a single locus in the genome of the mammalian cell by targeted integration.

In one embodiment of all independent aspects as well as of all dependent embodiments of the current invention the deoxyribonucleic acid encoding the trivalent, bispecific antibody is stably integrated into a single locus in the genome of the mammalian cell by single or double recombinase-mediate cassette exchange reaction.

In one dependent embodiment of each of the independent aspects as well as of all dependent embodiments according to the current invention the first heavy chain comprises in the CH3 domain the mutation T366W (numbering according to Kabat) and the second heavy chain comprises in the CH3 domain the mutations T366S, L368A, and Y407V (numbering according to Kabat), or vice versa.

In one dependent embodiment of each of the independent aspects as well as of all dependent embodiments according to the current invention one of the heavy chains further comprises the mutation S354C and the respective other heavy chain comprises the mutation Y349C (numbering according to Kabat).

In one dependent embodiment of each of the independent aspects as well as of all dependent embodiments according to the current invention the first heavy chain is an extended heavy chain comprising an additional domain exchanged Fab fragment.

In one dependent embodiment of each of the independent aspects as well as of all dependent embodiments according to the current invention the first light chain is a domain exchanged light chain VH-VL or CH1-CL.

In one dependent embodiment of each of the independent aspects as well as of all dependent embodiments according to the current invention
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one dependent embodiment of each of the independent aspects as well as of all dependent embodiments according to the current invention
the first heavy chain comprises from N- to C-terminus a first heavy chain variable domain, a CH1 domain, a second heavy chain variable domain, a CL domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a first light chain variable domain and a CH1 domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

In one dependent embodiment of each of the independent aspects as well as of all dependent embodiments according to the current invention the deoxyribonucleic acid is stably integrated into the genome of the mammalian cell at a single site or locus.

In one dependent embodiment of each of the independent aspects as well as of all dependent embodiments according to the current invention the deoxyribonucleic acid encoding the trivalent antibody comprises a further expression cassette encoding for a selection marker.

In one dependent embodiment of each of the independent aspects as well as of all dependent embodiments according to the current invention the expression cassette encoding for the selection marker is located partly 5′ and partly 3′ to the third recombination recognition sequence, wherein the 5′-located part of said expression cassette comprises the promoter and the start-codon and the 3′-located part of said expression cassette comprises the coding sequence without a start-codon and a polyA signal, wherein the start-codon is operably linked to the coding sequence.

In one dependent embodiment of each of the independent aspects as well as of all dependent embodiments according to the current invention the 5′-located part of the expression cassette encoding the selection marker comprises a promoter sequence operably linked to a start-codon, whereby the promoter sequence is flanked upstream by the third expression cassette and the start-codon is flanked downstream by the third recombination recognition sequence; and the 3′-located part of the expression cassette encoding the selection marker comprises a nucleic acid encoding the selection marker lacking a start-codon and is flanked upstream by the third recombination recognition sequence and downstream by the fourth expression cassette, wherein the start-codon is operably linked to the coding sequence.

In one dependent embodiment of each of the independent aspects as well as of all dependent embodiments according to the current invention
each expression cassette for an antibody chain comprises in 5′-to-3′ direction a promoter, a nucleic acid encoding an antibody chain, and a polyadenylation signal sequence and optionally a terminator sequence
and
each expression cassette encoding the selection marker comprises in 5′-to-3′ direction a promoter, a nucleic acid encoding the selection marker, and a polyadenylation signal sequence and optionally a terminator sequence.

In one dependent embodiment of each of the independent aspects as well as of all dependent embodiments according to the current invention the promoter is the human CMV promoter with intron A, the polyadenylation signal sequence is the bGH polyadenylation signal sequence and the terminator is the hGT terminator except for the expression cassette of the selection marker, wherein the promoter is the SV40 promoter and the polyadenylation signal sequence is the SV40 polyadenylation signal sequence and a terminator is absent.

In one dependent embodiment of each of the independent aspects as well as of all dependent embodiments according to the current invention the mammalian cell is a CHO cell.

In one dependent embodiment of each of the independent aspects as well as of all dependent embodiments according to the current invention in case the organization in 5′ to 3′-direction has as first expression cassette an expression cassette encoding the first heavy chain all cassettes are arranged unidirectional.

In one dependent embodiment of each of the independent aspects as well as of all dependent embodiments according to the current invention in case the organization in 5′ to 3′-direction is first expression cassette encoding the first light chain, second expression cassette encoding the first light chain, third expression cassette encoding the first heavy chain, fourth expression cassette encoding the second heavy chain, fifth expression cassette encoding the second light chain, sixth expression cassette encoding the second light chain, the first to third expression cassettes are arranged unidirectional and the fourth to sixth expression cassette are arranged unidirectional whereby the first to third expression cassettes are arranged in opposite direction of the fourth to sixth expression cassette.

In one dependent embodiment of each of the independent aspects as well as of all dependent embodiments according to the current invention the expression cassette encoding for a selection marker is located partly 5′ and partly 3′ to the third recombination recognition sequences, wherein the 5′-located part of said expression cassette comprises the promoter and a start-codon and the 3′-located part of said expression cassette comprises the coding sequence without a start-codon and a polyA signal.

In one dependent embodiment of each of the independent aspects as well as of all dependent embodiments according to the current invention the 5′-located part of the expression cassette encoding the selection marker comprises a promoter sequence operably linked to a start-codon, whereby the promoter sequence is flanked upstream by (i.e. is positioned downstream to) the second expression cassette and the start-codon is flanked downstream by (i.e. is positioned upstream of) the third recombination recognition sequence; and the 3′-located part of the expression cassette encoding the selection marker comprises a nucleic acid encoding the selection marker lacking a start-codon operably linked to a polyadenylation sequence and is flanked upstream by the third recombination recognition sequence and downstream by the third expression cassette.

In one dependent embodiment of each of the independent aspects as well as of all dependent embodiments according to the current invention the start-codon is a translation start-codon. In one embodiment the start-codon is ATG.

In one dependent embodiment of each of the independent aspects as well as of all dependent embodiments according to the current invention the first deoxyribonucleic acid is integrated into a first vector and the second deoxyribonucleic acid is integrated into a second vector.

In one dependent embodiment of each of the independent aspects as well as of all dependent embodiments according to the current invention each of the expression cassettes comprises in 5′-to-3′ direction a promoter, a coding sequence and a polyadenylation signal sequence optionally followed by a terminator sequence, which are all operably linked to each other.

In one dependent embodiment of each of the independent aspects as well as of all dependent embodiments according to the current invention the mammalian cell is a CHO cell. In one embodiment the CHO cell is a CHO-K1 cell.

In one dependent embodiment of each of the independent aspects as well as of all dependent embodiments according to the current invention the recombinase recognition sequences are L3, 2L and LoxFas. In one embodiment L3 has the sequence of SEQ ID NO: 01, 2L has the sequence of SEQ ID NO: 02 and LoxFas has the sequence of SEQ ID NO: 03. In one embodiment the first recombinase recognition sequence is L3, the second recombinase recognition sequence is 2L and the third recombinase recognition sequence is LoxFas.

In one dependent embodiment of each of the independent aspects as well as of all dependent embodiments according to the current invention the promoter is the human CMV promoter with intron A, the polyadenylation signal sequence is the bGH polyA site and the terminator sequence is the hGT terminator.

In one dependent embodiment of each of the independent aspects as well as of all dependent embodiments according to the current invention the promoter is the human CMV promoter with intron A, the polyadenylation signal sequence is the bGH polyA site and the terminator sequence is the hGT terminator except for the expression cassette(s) of the selection marker(s), wherein the promoter is the SV40 promoter and the polyadenylation signal sequence is the SV40 polyA site and a terminator sequence is absent.

In one dependent embodiment of each of the independent aspects as well as of all dependent embodiments according to the current invention the human CMV promoter has the sequence of SEQ ID NO: 04. In one embodiment the human CMV promoter has the sequence of SEQ ID NO: 06.

In one dependent embodiment of each of the independent aspects as well as of all dependent embodiments according to the current invention the bGH polyadenylation signal sequence is SEQ ID NO: 08.

In one dependent embodiment of each of the independent aspects as well as of all dependent embodiments according to the current invention the hGT terminator has the sequence of SEQ ID NO: 09.

In one dependent embodiment of each of the independent aspects as well as of all dependent embodiments according to the current invention the SV40 promoter has the sequence of SEQ ID NO: 10.

In one dependent embodiment of each of the independent aspects as well as of all dependent embodiments according to the current invention the SV40 polyadenylation signal sequence is SEQ ID NO: 07.

In one embodiment of all aspects and embodiments according to the current invention the trivalent antibody is a therapeutic antibody.

In one embodiment of all aspects and embodiments according to the current invention the trivalent, bispecific (therapeutic) antibody (TCB) comprises
a first and a second Fab fragment, wherein each binding site of the first and the second Fab fragment specifically bind to the second antigen,
a third Fab fragment, wherein the binding site of the third Fab fragment specifically binds to the first antigen, and wherein the third Fab fragment comprises a domain crossover such that the variable light chain domain (VL) and the variable heavy chain domain (VH) are replaced by each other, and
an Fc-region comprising a first Fc-region polypeptide and a second Fc-region polypeptide,
wherein the first and the second Fab fragment each comprise a heavy chain fragment and a full length light chain,
wherein the C-terminus of the heavy chain fragment of the first Fab fragment is fused to the N-terminus of the first Fc-region polypeptide,
wherein the C-terminus of the heavy chain fragment of the second Fab fragment is fused to the N-terminus of the variable light chain domain of the third Fab fragment and the C-terminus of the heavy chain constant domain 1 of the third Fab fragment is fused to the N-terminus of the second Fc-region polypeptide.

In one embodiment of all aspects and embodiments according to the current invention the trivalent antibody is an anti-CD3/CD20 bispecific antibody. In one embodiment the anti-CD3/CD20 bispecific antibody is a TCB with CD20 being the second antigen. In one embodiment the bispecific anti-CD3/CD20 antibody is RG6026. Such an antibody is reported in WO 2016/020309, which is incorporated herein by reference in its entirety.

In one embodiment of all aspects and embodiments according to the current invention the trivalent antibody is an anti-CD3/CEA bispecific antibody. In one embodiment the anti-CD3/CEA bispecific antibody is a TCB with CEA being the second antigen. In one embodiment the bispecific anti-CD3/CEA antibody is R06958688 or RG7802 or cibisatamab. Such an antibody is reported in WO 2017/055389, which is incorporated herein by reference in its entirety.

II.b Recombinase Mediated Cassette Exchange (RMCE)

Targeted integration allows for exogenous nucleotide sequences to be integrated into a pre-determined site of a mammalian cell's genome. In certain embodiments, the targeted integration is mediated by a recombinase that recognizes one or more recombination recognition sequences (RRSs). In certain embodiments, the targeted integration is mediated by homologous recombination.

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

In certain embodiments, a RRS is selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence. If multiple RRSs have to be present, the selection of each of the sequences is dependent on the other insofar as non-identical RRSs are chosen.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a φC31 integrase.

In certain embodiments when the RRS is a LoxP site, the cell requires the Cre recombinase to perform the recombination. In certain embodiments when the RRS is a FRT site, the cell requires the FLP recombinase to perform the recombination. In certain embodiments when the RRS is a Bxb1 attP or a Bxb1 attB site, the cell requires the Bxb1 integrase to perform the recombination. In certain embodiments when the RRS is a φC31 attP or a φC31attB site, the cell requires the (pC31 integrase to perform the recombination. The recombinases can be introduced into a cell using an expression vector comprising coding sequences of the enzymes.

The Cre-LoxP site-specific recombination system has been widely used in many biological experimental systems. Cre is a 38-kDa site-specific DNA recombinase that recognizes 34 bp LoxP sequences. Cre is derived from bacteriophage P1 and belongs to the tyrosine family site-specific recombinase. Cre recombinase can mediate both intra and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre-mediated recombination will excise DNA sequences located between the two LoxP sequences as a covalently closed circle. If two LoxP sequences are placed in an inverted position on the same nucleotide sequence, Cre-mediated recombination will invert the orientation of the DNA sequences located between the two sequences. If two LoxP sequences are on two different DNA molecules and if one DNA molecule is circular, Cre-mediated recombination will result in integration of the circular DNA sequence.

In certain embodiments, a LoxP sequence is a wild-type LoxP sequence. In certain embodiments, a LoxP sequence is a mutant LoxP sequence. Mutant LoxP sequences have been developed to increase the efficiency of Cre-mediated integration or replacement. In certain embodiments, a mutant LoxP sequence is selected from the group consisting of a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, and a Lox66 sequence. For example, the Lox71 sequence has 5 bp mutated in the left 13 bp repeat. The Lox66 sequence has 5 bp mutated in the right 13 bp repeat. Both the wild-type and the mutant LoxP sequences can mediate Cre-dependent recombination.

The term “matching RRSs” indicates that a recombination occurs between two RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

II.c Exemplary Mammalian Cells Suitable for TI

Any known or future mammalian cell suitable for TI comprising an exogenous nucleic acid (“landing site”) as described above can be used in the current invention.

The invention is exemplified with a CHO cell comprising an exogenous nucleic acid (landing site) according to the previous sections. This is presented solely to exemplify the invention but shall not be construed in any way as limitation. The true scope of the invention is set in the claims.

In one preferred embodiment the mammalian cell comprising an exogenous nucleotide sequence integrated at a single site within a locus of the genome of the mammalian cell is a CHO cell.

An exemplary mammalian cell comprising an exogenous nucleotide sequence integrated at a single site within a locus of its genome that is suitable for use in the current invention is a CHO cell harboring a landing site (=exogenous nucleotide sequence integrated at a single site within a locus of the genome of the mammalian cell) comprising three heterospecific loxP sites for Cre recombinase mediated DNA recombination. These heterospecific loxP sites are L3, LoxFas and 2L (see e.g. Lanza et al., Biotechnol. J. 7 (2012) 898-908; Wong et al., Nucleic Acids Res. 33 (2005) e147), whereby L3 and 2L flank the landing site at the 5′-end and 3′-end, respectively, and LoxFas is located between the L3 and 2L sites. The landing site further contains a bicistronic unit linking the expression of a selection marker via an IRES to the expression of the fluorescent GFP protein allowing to stabilize the landing site by positive selection as well as to select for the absence of the site after transfection and Cre-recombination (negative selection). Green fluorescence protein (GFP) serves for monitoring the RMCE reaction. An exemplary GFP has the sequence of SEQ ID NO: 11.

Such a configuration of the landing site as outlined in the previous paragraph allows for the simultaneous integration of two vectors, a so called front vector with an L3 and a LoxFas site and a back vector harboring a LoxFas and an 2L site. The functional elements of a selection marker gene different from that present in the landing site are distributed between both vectors: promoter and start codon are located on the front vector whereas coding region and poly A signal are located on the back vector. Only correct Cre-mediated integration of said nucleic acids from both vectors induces resistance against the respective selection agent.

Generally, a mammalian cell suitable for TI is a mammalian cell comprising an exogenous nucleotide sequence integrated at a single site within a locus of the genome of the mammalian cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different. Said exogenous nucleotide sequence is called a “landing site”.

The presently disclosed subject matter uses a mammalian cell suitable for TI of exogenous nucleotide sequences. In certain embodiments, the mammalian cell suitable for TI comprises an exogenous nucleotide sequence integrated at an integration site in the genome of the mammalian cell. Such a mammalian cell suitable for TI can be denoted also as a TI host cell.

In certain embodiments, the mammalian cell suitable for TI is a hamster cell, a human cell, a rat cell, or a mouse cell comprising a landing site. In certain embodiments, the mammalian cell suitable for TI is a Chinese hamster ovary (CHO) cell, a CHO K1 cell, a CHO K1SV cell, a CHO DG44 cell, a CHO DUKXB-11 cell, a CHO K1S cell, or a CHO KM cell comprising a landing site.

In certain embodiments, a mammalian cell suitable for TI comprises an integrated exogenous nucleotide sequence, wherein the exogenous nucleotide sequence comprises one or more recombination recognition sequence (RRS). In certain embodiments, the exogenous nucleotide sequence comprises at least two RRSs. The RRS can be recognized by a recombinase, for example, a Cre recombinase, an FLP recombinase, a Bxb1 integrase, or a φC31 integrase. The RRS can be selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

In certain embodiments, the exogenous nucleotide sequence comprises a first, a second and a third RRS, and at least one selection marker located between the first and the second RRS, and the third RRS is different from the first and/or the second RRS. In certain embodiments, the exogenous nucleotide sequence further comprises a second selection marker, and the first and the second selection markers are different. In certain embodiments, the exogenous nucleotide sequence further comprises a third selection marker and an internal ribosome entry site (IRES), wherein the IRES is operably linked to the third selection marker. The third selection marker can be different from the first or the second selection marker.

The selection marker(s) can be selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. The selection marker(s) can also be a fluorescent protein selected from the group consisting of green fluorescent protein (GFP), enhanced GFP (eGFP), a synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald6, CyPet, mCFPm, Cerulean, and T-Sapphire.

In certain embodiments, the exogenous nucleotide sequence comprises a first, second, and third RRS, and at least one selection marker located between the first and the third RRS.

An exogenous nucleotide sequence is a nucleotide sequence that does not originate from a specific cell but can be introduced into said cell by DNA delivery methods, such as, e.g., by transfection, electroporation, or transformation methods. In certain embodiments, a mammalian cell suitable for TI comprises at least one exogenous nucleotide sequence integrated at one or more integration sites in the mammalian cell's genome. In certain embodiments, the exogenous nucleotide sequence is integrated at one or more integration sites within a specific locus of the genome of the mammalian cell.

In certain embodiments, an integrated exogenous nucleotide sequence comprises one or more recombination recognition sequence (RRS), wherein the RRS can be recognized by a recombinase. In certain embodiments, the integrated exogenous nucleotide sequence comprises at least two RRSs. In certain embodiments, an integrated exogenous nucleotide sequence comprises three RRSs, wherein the third RRS is located between the first and the second RRS. In certain embodiments, the first and the second RRS are the same and the third RRS is different from the first or the second RRS. In certain preferred embodiments, all three RRSs are different. In certain embodiments, the RRSs are selected independently of each other from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

In certain embodiments, the integrated exogenous nucleotide sequence comprises at least one selection marker. In certain embodiments, the integrated exogenous nucleotide sequence comprises a first, a second and a third RRS, and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker.

In certain embodiments, a selection marker is located between a first and a second RRS and the two flanking RRSs are different. In certain preferred embodiments, the first flanking RRS is a LoxP L3 sequence and the second flanking RRS is a LoxP 2L sequence. In certain embodiments, a LoxP L3 sequenced is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence. In certain embodiments, the first flanking RRS is a φC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientation.

In certain embodiments, the integrated exogenous nucleotide sequence comprises a first and a second selection marker, which are flanked by two RRSs, wherein the first selection marker is different from the second selection marker. In certain embodiments, the two selection markers are both independently of each other selected from the group consisting of a glutamine synthetase selection marker, a thymidine kinase selection marker, a HYG selection marker, and a puromycin resistance selection marker. In certain embodiments, the integrated exogenous nucleotide sequence comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire fluorescent protein. In certain embodiments, the first selection marker is a glutamine synthetase selection marker and the second selection marker is a GFP fluorescent protein. In certain embodiments, the two RRSs flanking both selection markers are different.

In certain embodiments, the selection marker is operably linked to a promoter sequence. In certain embodiments, the selection marker is operably linked to an SV40 promoter. In certain embodiments, the selection marker is operably linked to a human Cytomegalovirus (CMV) promoter.

In certain embodiments, the integrated exogenous nucleotide sequence comprises three RRSs. In certain embodiments, the third RRS is located between the first and the second RRS. In certain embodiments, the first and the second RRS are the same, and the third RRS is different from the first or the second RRS. In certain preferred embodiments, all three RRSs are different.

II.d Exemplary Vectors Suitable for Performing the Invention

Beside the “single-vector RMCE” as outlined above a novel “two-vector RMCE” can be performed for simultaneous targeted integration of two nucleic acids.

A “two-vector RMCE” strategy is employed in the method according to the current invention using a vector combination according to the current invention. For example, but not by way of limitation, an integrated exogenous nucleotide sequence could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence. An example of a two vector RMCE strategy is illustrated in FIG. 1. Such two vector RMCE strategies allow for the introduction of multiple SOIs by incorporating the appropriate number of SOIs in the respective sequence between each pair of RRSs so that the expression cassette organization according to the current invention is obtained after TI in the genome of the mammalian cell suitable for TI.

The two-plasmid RMCE strategy involves using three RRS sites to carry out two independent RMCEs simultaneously (FIG. 1). Therefore, a landing site in the mammalian cell suitable for TI using the two-plasmid RMCE strategy includes a third RRS site (RRS3) that has no cross activity with either the first RRS site (RRS1) or the second RRS site (RRS2). The two expression plasmids to be targeted require the same flanking RRS sites for efficient targeting, one expression plasmid (front) flanked by RRS1 and RRS3 and the other (back) by RRS3 and RRS2. Also two selection markers are needed in the two-plasmid RMCE. One selection marker expression cassette was split into two parts. The front plasmid would contain the promoter followed by a start codon and the RRS3 sequence. The back plasmid would have the RRS3 sequence fused to the N-terminus of the selection marker coding region, minus the start-codon (ATG). Additional nucleotides may need to be inserted between the RRS3 site and the selection marker sequence to ensure in frame translation for the fusion protein, i.e. operable linkage. Only when both plasmids are correctly inserted the full expression cassette of the selection marker will be assembled and, thus, rendering cells resistance to the respective selection agent. FIG. 1 is the schematic diagram showing the two plasmid RMCE strategy.

Both single-vector and two-vector RMCE allow for unidirectional integration of one or more donor DNA molecule(s) into a pre-determined site of a mammalian cell's genome by precise exchange of a DNA sequence present on the donor DNA with a DNA sequence in the mammalian cell's genome where the integration site resides. These DNA sequences are characterized by two heterospecific RRSs flanking i) at least one selection marker or as in certain two-vector RMCEs a “split selection marker”; and/or ii) at least one exogenous SOI.

RMCE involves double recombination cross-over events, catalyzed by a recombinase, between the two heterospecific RRSs within the target genomic locus and the donor DNA molecule. RMCE is designed to introduce a copy of the DNA sequences from the front- and back-vector in combination into the pre-determined locus of a mammalian cell's genome. Unlike recombination which involves just one cross-over event, RMCE can be implemented such that prokaryotic vector sequences are not introduced into the mammalian cell's genome, thus reducing and/or preventing unwanted triggering of host immune or defense mechanisms. The RMCE procedure can be repeated with multiple DNA sequences.

In certain embodiments, targeted integration is achieved by two RMCEs, wherein two different DNA sequences, each comprising at least one expression cassette encoding a part of a heteromultimeric polypeptide and/or at least one selection marker or part thereof flanked by two heterospecific RRSs, are both integrated into a pre-determined site of the genome of a mammalian cell suitable for TI. In certain embodiments, targeted integration is achieved by multiple RMCEs, wherein DNA sequences from multiple vectors, each comprising at least one expression cassette encoding a part of a heteromultimeric polypeptide and/or at least one selection marker or part thereof flanked by two heterospecific RRSs, are all integrated into a predetermined site of the genome of a mammalian cell suitable for TI. In certain embodiments the selection marker can be partially encoded on the first the vector and partially encoded on the second vector such that only the correct integration of both by double RMCE allows for the expression of the selection marker. An example of such a system is presented in FIG. 1.

In certain embodiments, targeted integration via recombinase-mediated recombination leads to selection marker and/or the different expression cassettes for the multimeric polypeptide integrated into one or more pre-determined integration sites of a host cell genome free of sequences from a prokaryotic vector.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

The following examples and figures are provided to aid the understanding of the present invention, the true scope of which is set forth in the appended claims.

### DESCRIPTION OF THE FIGURES

FIG. 1: Scheme of a two-plasmid RMCE strategy involving the use of three RRS sites to carry out two independent RMCEs simultaneously.

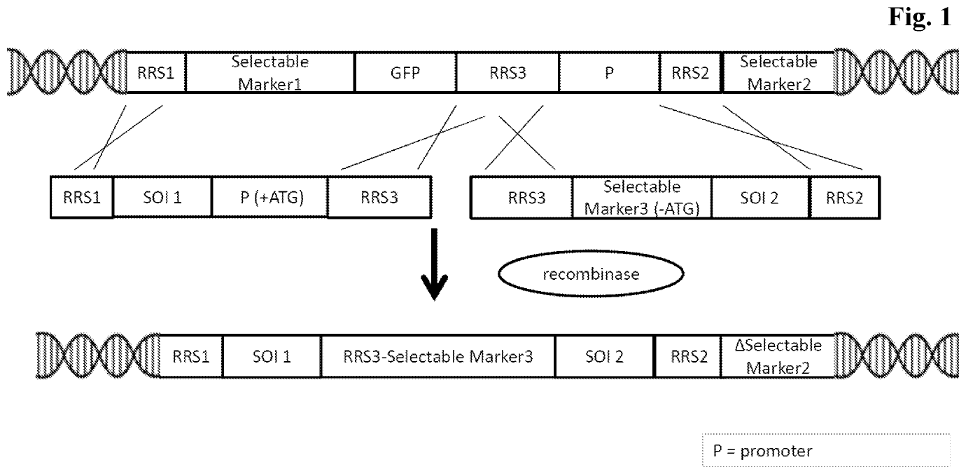

### DESCRIPTION OF THE SEQUENCES

SEQ ID NO: 01: exemplary sequence of an L3 recombinase recognition sequence

SEQ ID NO: 02: exemplary sequence of a 2L recombinase recognition sequence

SEQ ID NO: 03: exemplary sequence of a LoxFas recombinase recognition sequence

SEQ ID NO: 04-06: exemplary variants of human CMV promoter

SEQ ID NO: 07: exemplary SV40 polyadenylation signal sequence

SEQ ID NO: 08: exemplary bGH polyadenylation signal sequence

SEQ ID NO: 09: exemplary hGT terminator sequence

SEQ ID NO: 10: exemplary SV40 promoter sequence

SEQ ID NO: 11: exemplary GFP nucleic acid sequence

### EXAMPLES

### Example 1

General Techniques

1) Recombinant DNA Techniques

Standard methods were used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents were used according to the manufacturer's instructions.

2) DNA Sequence Determination

DNA sequencing was performed at SequiServe GmbH (Vaterstetten, Germany)

3) DNA and Protein Sequence Analysis and Sequence Data Management

The EMBOSS (European Molecular Biology Open Software Suite) software package and Invitrogen's Vector NTI version 11.5 or Geneious Prime were used for sequence creation, mapping, analysis, annotation and illustration.

4) Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

5) Reagents

All commercial chemicals, antibodies and kits were used as provided according to the manufacturer's protocol if not stated otherwise.

6) Cultivation of TI Host Cell Line

TI CHO host cells were cultivated at 37° C. in a humidified incubator with 85% humidity and 5% CO2. They were cultivated in a proprietary DMEM/F12-based medium containing 300 μg/ml Hygromycin B and 4 μg/ml of a second selection marker. The cells were splitted every 3 or 4 days at a concentration of 0.3×10E6 cells/ml in a total volume of 30 ml. For the cultivation 125 ml non-baffle Erlenmeyer shake flasks were used. Cells were shaken at 150 rpm with a shaking amplitude of 5 cm. The cell count was determined with Cedex HiRes Cell Counter (Roche). Cells were kept in culture until they reached an age of 60 days.

7) Cloning

General

Cloning with R-sites depends on DNA sequences next to the gene of interest (SOI) that are equal to sequences lying in following fragments. Like that, assembly of fragments is possible by overlap of the equal sequences and subsequent sealing of nicks in the assembled DNA by a DNA ligase. Therefore, a cloning of the single genes in particular preliminary vectors containing the right R-sites is necessary. After successful cloning of these preliminary vectors the gene of interest flanked by the R-sites is cut out via restriction digest by enzymes cutting directly next to the R-sites. The last step is the assembly of all DNA fragments in one step. In more detail, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some vectors, a cloning strategy via restriction enzymes was used. By selection of suitable restriction enzymes, the wanted gene of interest can be cut out and afterwards inserted into a different vector by ligation. Therefore, enzymes cutting in a multiple cloning site (MCS) are preferably used and chosen in a smart manner, so that a ligation of the fragments in the correct array can be conducted. If vector and fragment are previously cut with the same restriction enzyme, the sticky ends of fragment and vector fit perfectly together and can be ligated by a DNA ligase, subsequently. After ligation, competent E. coli cells are transformed with the newly generated plasmid.

Cloning Via Restriction Digestion

For the digest of plasmids with restriction enzymes the following components were pipetted together on ice:

TABLE
Restriction Digestion Reaction Mix

| component | ng (set point) | μl |
|---|---|---|
| purified DNA | tbd | tbd |
| CutSmart Buffer (10x) | | 5 |
| Restriction Enzyme | | 1 |
| PCR-grade Water | | ad 50 |
| Total | | 50 |

If more enzymes were used in one digestion, 1 μl of each enzyme was used and the volume adjusted by addition of more or less PCR-grade water. All enzymes were selected on the preconditions that they are qualified for the use with CutSmart buffer from new England Biolabs (100% activity) and have the same incubation temperature (all 37° C.).

Incubation was performed using thermomixers or thermal cyclers, allowing to incubate the samples at a constant temperature (37° C.). During incubation the samples were not agitated. Incubation time was set at 60 min. Afterwards the samples were directly mixed with loading dye and loaded onto an agarose electrophoresis gel or stored at 4° C./on ice for further use.

A 1% agarose gel was prepared for gel electrophoresis. Therefor 1.5 g of multi-purpose agarose were weighed into a 125 Erlenmeyer shake flask and filled up with 150 ml TAE-buffer. The mixture was heated up in a microwave oven until the agarose was completely dissolved. 0.5 μg/ml ethidium bromide were added into the agarose solution. Thereafter the gel was cast in a mold. After the agarose was set, the mold was placed into the electrophoresis chamber and the chamber filled with TAE-buffer. Afterwards the samples were loaded. In the first pocket (from the left) an appropriate DNA molecular weight marker was loaded, followed by the samples. The gel was run for around 60 minutes at <130V. After electrophoresis the gel was removed from the chamber and analyzed in an UV-Imager.

The target bands were cut and transferred to 1.5 ml Eppendorf tubes. For purification of the gel, the QIAquick Gel Extraction Kit from Qiagen was used according to the manufacturer's instructions. The DNA fragments were stored at −20° C. for further use.

The fragments for the ligation were pipetted together in a molar ratio of 1:2, 1:3 or 1:5 vector to insert, depending on the length of the inserts and the vector-fragments and their correlation to each other. If the fragment, that should be inserted into the vector was short, a 1:5-ratio was used. If the insert was longer, a smaller amount of it was used in correlation to the vector. An amount of 50 ng of vector were used in each ligation and the particular amount of insert calculated with NEBioCalculator. For ligation, the T4 DNA ligation kit from NEB was used. An example for the ligation mixture is depicted in the following Table:

TABLE
Ligation Reaction Mix

| component | ng (set point) | conc. [ng/μl] | μl |
|---|---|---|---|
| T4 DNA Ligase Buffer (10x) | | | 2 |
| Vector DNA (4000 bp) | 50 | 50 | 1 |
| Insert DNA (2000 bp) | 125 | 20 | 6.25 |
| Nuclease-free Water | | | 9.75 |
| T4 Ligase | | | 1 |
| Total | | | 20 |

All components were pipetted together on ice, starting with the mixing of DNA and water, addition of buffer and finally addition of the enzyme. The reaction was gently mixed by pipetting up and down, briefly microfuged and then incubated at room temperature for 10 minutes. After incubation, the T4 ligase was heat inactivated at 65° C. for 10 minutes. The sample was chilled on ice. In a final step, 10-beta competent E. coli cells were transformed with 2 μl of the ligated plasmid (see below).

Cloning Via R-Site Assembly

For assembly, all DNA fragments with the R-sites at each end were pipetted together on ice. An equimolar ratio (0.05 ng) of all fragments was used, as recommended by the manufacturer, when more than 4 fragments are being assembled. One half of the reaction mix was embodied by NEBuilder HiFi DNA Assembly Master Mix. The total reaction volume was 40 μl and was reached by a fill-up with PCR-clean water. In the following Table an exemplary pipetting scheme is depicted.

TABLE
Assembly Reaction Mix

| | | pmol | ng | conc. | |
|---|---|---|---|---|---|
| component | bp | (set point) | (set point) | [ng/μl] | μl |
| Insert 1 | 2800 | 0.05 | 88.9 | 21 | 4.23 |
| Insert 2 | 2900 | 0.05 | 90.5 | 35 | 2.59 |
| Insert 3 | 4200 | 0.05 | 131.6 | 35.5 | 3.71 |
| Insert 4 | 3600 | 0.05 | 110.7 | 23 | 4.81 |
| Vector | 4100 | 0.05 | 127.5 | 57.7 | 2.21 |
| NEBuilder HiFi DNA | | | | | 20 |
| Assembly Master Mix | | | | | |
| PCR-clean Water | | | | | 2.45 |
| Total | | | | | 40 |

After set up of the reaction mixture, the tube was incubated in a thermocycler at constantly 50° C. for 60 minutes. After successful assembly, 10-beta competent E. coli bacteria were transformed with 2 μl of the assembled plasmid DNA (see below).

Transformation 10-Beta Competent E. coli Cells

For transformation the 10-beta competent E. coli cells were thawed on ice. After that, 2 μl of plasmid DNA were pipetted directly into the cell suspension. The tube was flicked and put on ice for 30 minutes. Thereafter, the cells were placed into the 42° C.-warm thermal block and heat-shocked for exactly 30 seconds. Directly afterwards, the cells were chilled on ice for 2 minutes. 950 μl of NEB 10-beta outgrowth medium were added to the cell suspension. The cells were incubated under shaking at 37° C. for one hour. Then, 50-100 μl were pipetted onto a pre-warmed (37° C.) LB-Amp agar plate and spread with a disposable spatula. The plate was incubated overnight at 37° C. Only bacteria which have successfully incorporated the plasmid, carrying the resistance gene against ampicillin, can grow on this plates. Single colonies were picked the next day and cultured in LB-Amp medium for subsequent plasmid preparation.

Bacterial Culture

Cultivation of E. coli was done in LB-medium, short for Luria Bertani, that was spiked with 1 ml/L 100 mg/ml ampicillin resulting in an ampicillin concentration of 0.1 mg/ml. For the different plasmid preparation quantities, the following amounts were inoculated with a single bacterial colony.

TABLE
E. coli cultivation volumes

| | Volume LB-Amp | Incubation time |
|---|---|---|
| Quantity plasmid preparation | medium [ml] | [h] |
| Mini-Prep 96-well (EpMotion) | 1.5 | 23 |
| Mini-Prep 15 ml-tube | 3.6 | 23 |
| Maxi-Prep | 200 | 16 |

For Mini-Prep, a 96-well 2 ml deep-well plate was filled with 1.5 ml LB-Amp medium per well. The colonies were picked and the toothpick was tuck in the medium. When all colonies were picked, the plate closed with a sticky air porous membrane. The plate was incubated in a 37° C. incubator at a shaking rate of 200 rpm for 23 hours.

For Mini-Preps a 15 ml-tube (with a ventilated lid) was filled with 3.6 ml LB-Amp medium and equally inoculated with a bacterial colony. The toothpick was not removed but left in the tube during incubation. Like the 96-well plate the tubes were incubated at 37° C., 200 rpm for 23 hours.

For Maxi-Prep 200 ml of LB-Amp medium were filled into an autoclaved glass 1 L Erlenmeyer flask and inoculated with 1 ml of bacterial day-culture, that was roundabout 5 hours old. The Erlenmeyer flask was closed with a paper plug and incubated at 37° C., 200 rpm for 16 hours.

Plasmid Preparation

For Mini-Prep, 50 μl of bacterial suspension were transferred into a 1 ml deep-well plate. After that, the bacterial cells were centrifuged down in the plate at 3000 rpm, 4° C. for 5 min. The supernatant was removed and the plate with the bacteria pellets placed into an EpMotion. After ca. 90 minutes the run was done and the eluted plasmid-DNA could be removed from the EpMotion for further use.

For Mini-Prep, the 15 ml tubes were taken out of the incubator and the 3.6 ml bacterial culture splitted into two 2 ml Eppendorf tubes. The tubes were centrifuged at 6,800×g in a table-top microcentrifuge for 3 minutes at room temperature. After that, Mini-Prep was performed with the Qiagen QIAprep Spin Miniprep Kit according to the manufacturer's instructions. The plasmid DNA concentration was measured with Nanodrop.

Maxi-Prep was performed using the Macherey-Nagel NucleoBond® Xtra Maxi EF Kit according to the manufacturer's instructions. The DNA concentration was measured with Nanodrop.

Ethanol Precipitation

The volume of the DNA solution was mixed with the 2.5-fold volume ethanol 100%. The mixture was incubated at −20° C. for 10 min. Then the DNA was centrifuged for 30 min. at 14,000 rpm, 4° C. The supernatant was carefully removed and the pellet washed with 70% ethanol. Again, the tube was centrifuged for 5 min. at 14,000 rpm, 4° C. The supernatant was carefully removed by pipetting and the pellet dried. When the ethanol was evaporated, an appropriate amount of endotoxin-free water was added. The DNA was given time to re-dissolve in the water overnight at 4° C. A small aliquot was taken and the DNA concentration was measured with a Nanodrop device.

### Example 2

Plasmid Generation

Expression Cassette Composition

For the expression of an antibody chain a transcription unit comprising the following functional elements was used:
the immediate early enhancer and promoter from the human cytomegalovirus including intron A,
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA), and
optionally the human gastrin terminator (hGT).

Beside the expression unit/cassette including the desired gene to be expressed the basic/standard mammalian expression plasmid contains
an origin of replication from the vector pUC18 which allows replication of this plasmid in E. coli, and
a beta-lactamase gene which confers ampicillin resistance in E. coli.

Front- and Back-Vector Cloning

To construct two-plasmid antibody constructs, antibody HC and LC fragments were cloned into a front vector backbone containing L3 and LoxFAS sequences, and a back vector containing LoxFAS and 2L sequences and a pac selectable marker. The Cre recombinase plasmid pOG231 (Wong, E. T., et al., Nucleic Acids Res 2005, 33, (17), e147; O'Gorman S et al., Proc. Natl. Acad. Sci. USA 1997, 94, (26), 14602-7) was used for all RMCE processes.

The cDNAs encoding the respective antibody chains were generated by gene synthesis (Geneart, Life Technologies Inc.). The gene synthesis and the backbone-vectors were digested with HindIII-HF and EcoRI-HF (NEB) at 37° C. for 1 h and separated by agarose gel electrophoresis. The DNA-fragment of the insert and backbone were cut out from the agarose gel and extracted by QIAquick Gel Extraction Kit (Qiagen). The purified insert and backbone fragment was ligated via the Rapid Ligation Kit (Roche) following the manufacturer's protocol with an Insert/Backbone ratio of 3:1. The ligation approach was then transformed in competent E. coli DH5α via heat shock for 30 sec. at 42° C. and incubated for 1 h at 37° C. before they were plated out on agar plates with ampicillin for selection. Plates were incubated at 37° C. overnight.

On the following day clones were picked and incubated overnight at 37° C. under shaking for the Mini or Maxi-Preparation, which was performed with the EpMotion® 5075 (Eppendorf) or with the QIAprep Spin Mini-Prep Kit (Qiagen)/NucleoBond Xtra Maxi EF Kit (Macherey & Nagel), respectively. All constructs were sequenced to ensure the absence of any undesirable mutations (Sequi Serve GmbH).

In the second cloning step, the previously cloned vectors were digested with KpnI-HF/SalI-HF and SalI-HF/MfeI-HF with the same conditions as for the first cloning. The TI backbone vector was digested with KpnI-HF and MfeI-HF. Separation and extraction was performed as described above. Ligation of the purified insert and backbone was performed using T4 DNA Ligase (NEB) following the manufacturing protocol with an Insert/Insert/Backbone ratio of 1:1:1 overnight at 4° C. and inactivated at 65° C. for 10 min. The following cloning steps were performed as described above.

The cloned plasmids were used for the TI transfection and pool generation.

### Example 3

Cultivation, Transfection, Selection and Single Cell Cloning

TI host cells were propagated in disposable 125 ml vented shake flasks under standard humidified conditions (95% rH, 37° C., and 5% CO₂) at a constant agitation rate of 150 rpm in a proprietary DMEM/F12-based medium. Every 3-4 days the cells were seeded in chemically defined medium containing selection marker 1 and selection marker 2 in effective concentrations with a concentration of 3×10E5 cells/ml. Density and viability of the cultures were measured with a Cedex HiRes cell counter (F. Hoffmann-La Roche Ltd, Basel, Switzerland).

For stable transfection, equimolar amounts of front and back vector were mixed. 1 μg Cre expression plasmid was added to 5 μg of the mixture.

Two days prior to transfection TI host cells were seeded in fresh medium with a density of 4×10E5 cells/ml. Transfection was performed with the Nucleofector device using the Nucleofector Kit V (Lonza, Switzerland), according to the manufacturer's protocol. 3×10E7 cells were transfected with 30 μg plasmid. After transfection the cells were seeded in 30 ml medium without selection agents.

On day 5 after seeding the cells were centrifuged and transferred to 80 mL chemically defined medium containing puromycin (selection agent 1) and 1-(2′-deoxy-2′-fluoro-l-beta-D-arabinofuranosyl-5-iodo)uracil (FIAU; selection agent 2) at effective concentrations at 6×10E5 cells/ml for selection of recombinant cells. The cells were incubated at 37° C., 150 rpm. 5% CO2, and 85% humidity from this day on without splitting. Cell density and viability of the culture was monitored regularly. When the viability of the culture started to increase again, the concentrations of selection agents 1 and 2 were reduced to about half the amount used before. In more detail, to promote the recovering of the cells, the selection pressure was reduced if the viability is >40% and the viable cell density (VCD) is >0.5×10E6 cell s/mL. Therefore, 4×10E5 cells/ml were centrifuged and resuspended in 40 ml selection media II (chemically-defined medium, ½ selection marker 1 & 2). The cells were incubated with the same conditions as before and also not splitted.

Ten days after starting selection, the success of Cre mediated cassette exchange was checked by flow cytometry measuring the expression of intracellular GFP and extracellular trivalent antibody (TCB) bound to the cell surface. An APC antibody (allophycocyanin-labeled F(ab′)2 Fragment goat anti-human IgG) against human antibody light and heavy chain was used for FACS staining. Flow cytometry was performed with a BD FACS Canto II flow cytometer (BD, Heidelberg, Germany). Ten thousand events per sample were measured. Living cells were gated in a plot of forward scatter (FSC) against side scatter (SSC). The live cell gate was defined with non-transfected TI host cells and applied to all samples by employing the FlowJo 7.6.5 EN software (TreeStar, Olten, Switzerland). Fluorescence of GFP was quantified in the FITC channel (excitation at 488 nm, detection at 530 nm). Trivalent antibody (TCB) was measured in the APC channel (excitation at 645 nm, detection at 660 nm). Parental CHO cells, i.e. those cells used for the generation of the TI host cell, were used as a negative control with regard to GFP and trivalent antibody (TCB) expression. Fourteen days after the selection had been started, the viability exceeded 90% and selection was considered as complete.

### Example 4

FACS Screening

FACS analysis was performed to check the transfection efficiency and the RMCE efficiency of the transfection. 4×10E5 cells of the transfected approaches were centrifuged (1200 rpm, 4 min.) and washed twice with 1 mL PBS. After the washing steps with PBS the pellet was resuspended in 400 μL PBS and transferred in FACS tubes (Falcon® Round-Bottom Tubes with cell strainer cap; Corning). The measurement was performed with a FACS Canto II and the data were analyzed by the software FlowJo.

### Example 5

Fed-Batch Cultivation

Fed-batch production cultures were performed in shake flasks or Ambr15 vessels (Sartorius Stedim) with proprietary chemically defined medium. Cells were seeded at 1×10E6 cells/ml on day 0, with a temperature shift on day 3. Cultures received proprietary feed medium on days 3, 7, and 10. Viable cell count (VCC) and percent viability of cells in culture was measured on days 0, 3, 7, 10, and 14 using a Vi-Cell™ XR instrument (Beckman Coulter). Glucose and lactate concentrations were measured on days 7, 10 and 14 using a Bioprofile 400 Analyzer (Nova Biomedical). The supernatant was harvested 14 days after start of fed-batch by centrifugation (10 min, 1000 rpm and 10 min, 4000 rpm) and cleared by filtration (0.22 μm). Day 14 titers were determined using protein A affinity chromatography with UV detection.

Product quality was determined by Caliper's LabChip (Caliper Life Sciences).

### Example 6

Effect of Vector Design

To examine the effect of expression cassette organization on productivity in the TI host, RMCE pools were generated by transfecting two plasmids (front and back vector) containing different numbers and organizations of the individual chains of a trivalent antibody in the TCB format.

After selection, recovery, and verification of RMCE by flow cytometry, the pools' productivity was evaluated in a 14-day fed batch production assay. For specific vector organizations an increase in titer compared to the reference pools was observed.

The effect of the antibody chain expression cassette organization on expression of five different TCBs was evaluated. TCB 1 to 5 all had a different targeting specificity. TCB 3 was tested with 4 different anti-CD3 binding sites.

For TCB-1 the following results have been obtained; the reference organizations are shaded in grey:

For TCB-3 the following results have been obtained:

| | front vector | | | | back vector | | | | | | |
|---|---|---|---|---|---|---|---|---|---|---|---|
| | expression cassettes | | | | expression cassettes | | | | | % MP | eff. |
| TCB | in 5'-to 3' direction | | | | in 5'-to 3' direction | | | | titer | (CE- | Titer |
| No. | 1 | 2 | 3 | 4 | 1 | 2 | 3 | 4 | [g/L] | SDS) | [g/L] |
| 3-1 | k | l | l | — | xl | h | — | — | 3.5 | 95 | 3.33 |
| 3-1 | k | xl | l | — | xl | h | — | — | 3 | 95 | 2.85 |
| 3-1 | k | xl | l | — | h | l | l | — | 3.7 | 68 | 2.52 |
| 3-1 | k | xl | xl | — | h | l | l | — | 3 | 65 | 1.95 |
| 3-2 | k | 1 | l | — | xl | h | — | — | 2.95 | 95 | 2.8 |
| 3-2 | k | xl | l | — | xl | h | — | — | 2.6 | 93 | 2.42 |
| 3-2 | k | xl | l | — | h | l | l | — | 2.5 | 72 | 1.8 |
| 3-2 | k | xl | xl | — | h | l | l | — | 3.1 | 70 | 2.17 |
| 3-3 | k | xl | l | — | xl | h | — | — | 2.95 | 93 | 2.74 |
| 3-3 | k | l | l | — | xl | h | — | — | 2.6 | 93 | 2.42 |
| 3-3 | k | xl | l | — | h | l | l | — | 2.5 | 72 | 1.8 |
| 3-3 | k | xl | xl | — | h | l | l | — | 3.15 | 70 | 2.2 |
| 3-4 | k | l | l | — | xl | h | — | — | 3.35 | 95 | 3.18 |
| 3-4 | k | xl | l | — | xl | h | — | — | 2.75 | 95 | 2.61 |
| 3-4 | k | xl | l | — | h | l | l | — | 3.7 | 69 | 2.55 |
| 3-4 | k | xl | xl | — | h | l | l | — | 3.15 | 68 | 2.14 |

For TCB-2, -4 and -5 the following results have been obtained:

| | front vector | | | | back vector | | | | | | |
|---|---|---|---|---|---|---|---|---|---|---|---|
| | expression cassettes | | | | expression cassettes | | | | | % MP | eff. |
| TCB | in 5'-to 3' direction | | | | in 5'-to 3' direction | | | | titer | (CE- | Titer |
| No. | 1 | 2 | 3 | 4 | 1 | 2 | 3 | 4 | [g/L] | SDS) | [g/L] |
| 2 | k | xl | l | — | h | l | xl | — | 4.3 | 73 | 3.14 |
| 2 | k | xl | xl | — | h | l | l | — | 2.7 | 60 | 1.62 |
| 2 | k | xl | k | — | l | l | h | — | 0.9 | 74 | 0.67 |
| 4 | k | xl | xl | — | h | l | l | — | 2.16 | 76 | 1.64 |
| 4 | k | xl | xl | — | h | l | — | — | 1.02 | 19.5 | 0.81 |
| 5 | k | xl | xl | — | h | l | l | — | 3.6 | 76 | 2.74 |
| 5 | k | xl | xl | — | h | l | — | — | 1.8 | 59 | 1.06 |

MP=main product, eff. titer=effective titer=titer multiplied by % main product

1. A method for producing a trivalent antibody comprising the steps of
a) cultivating a mammalian cell comprising a deoxyribonucleic acid encoding the trivalent antibody, and
b) recovering the trivalent antibody from the cell or the cultivation medium,
wherein the deoxyribonucleic acid encoding the trivalent antibody is stably integrated into the genome of the mammalian cell and comprises in 5′- to 3′-direction
either (1)
a first expression cassette encoding the first heavy chain,
a second expression cassette encoding the first light chain,
a third expression cassette encoding the first light chain,
a fourth expression cassette encoding the second heavy chain, and
a fifth expression cassette encoding the second light chain,
or (2)
a first expression cassette encoding the first heavy chain,
a second expression cassette encoding the first light chain,
a third expression cassette encoding the first light chain,
a fourth expression cassette encoding the second heavy chain,
a fifth expression cassette encoding the second light chain,
a sixth expression cassette encoding the second light chain,
or (3)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second light chain,
or (4)
a first expression cassette encoding the first heavy chain,
a second expression cassette encoding the second heavy chain,
a third expression cassette encoding the first light chain,
a fourth expression cassette encoding the second light chain,
a fifth expression cassette encoding the first heavy chain,
a sixth expression cassette encoding the second light chain,
or (5)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second heavy chain,
a sixth expression cassette encoding the second light chain,
a seventh expression cassette encoding the second light chain,
or (6)
a first expression cassette encoding the first light chain,
a second expression cassette encoding the first light chain,
a third expression cassette encoding the first heavy chain,
a fourth expression cassette encoding the second heavy chain,
a fifth expression cassette encoding the second light chain,
a sixth expression cassette encoding the second light chain.

2. A deoxyribonucleic acid encoding a trivalent antibody comprising in 5′- to 3′-direction
either (1)
a first expression cassette encoding the first heavy chain,
a second expression cassette encoding the first light chain,
a third expression cassette encoding the first light chain,
a fourth expression cassette encoding the second heavy chain, and
a fifth expression cassette encoding the second light chain,
or (2)
a first expression cassette encoding the first heavy chain,
a second expression cassette encoding the first light chain,
a third expression cassette encoding the first light chain,
a fourth expression cassette encoding the second heavy chain,
a fifth expression cassette encoding the second light chain,
a sixth expression cassette encoding the second light chain,
or (3)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second light chain,
or (4)
a first expression cassette encoding the first heavy chain,
a second expression cassette encoding the second heavy chain,
a third expression cassette encoding the first light chain,
a fourth expression cassette encoding the second light chain,
a fifth expression cassette encoding the first heavy chain,
a sixth expression cassette encoding the second light chain,
or (5)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second heavy chain,
a sixth expression cassette encoding the second light chain,
a seventh expression cassette encoding the second light chain,
or (6)
a first expression cassette encoding the first light chain,
a second expression cassette encoding the first light chain,
a third expression cassette encoding the first heavy chain,
a fourth expression cassette encoding the second heavy chain,
a fifth expression cassette encoding the second light chain,
a sixth expression cassette encoding the second light chain.

3. Use of a deoxyribonucleic acid comprising in 5′- to 3′-direction
either (1)
a first expression cassette encoding the first heavy chain,
a second expression cassette encoding the first light chain,
a third expression cassette encoding the first light chain,
a fourth expression cassette encoding the second heavy chain, and
a fifth expression cassette encoding the second light chain,
or (2)
a first expression cassette encoding the first heavy chain,
a second expression cassette encoding the first light chain,
a third expression cassette encoding the first light chain,
a fourth expression cassette encoding the second heavy chain,
a fifth expression cassette encoding the second light chain,
a sixth expression cassette encoding the second light chain,
or (3)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second light chain,
or (4)
a first expression cassette encoding the first heavy chain,
a second expression cassette encoding the second heavy chain,
a third expression cassette encoding the first light chain,
a fourth expression cassette encoding the second light chain,
a fifth expression cassette encoding the first heavy chain,
a sixth expression cassette encoding the second light chain,
or (5)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second heavy chain,
a sixth expression cassette encoding the second light chain,
a seventh expression cassette encoding the second light chain,
or (6)
a first expression cassette encoding the first light chain,
a second expression cassette encoding the first light chain,
a third expression cassette encoding the first heavy chain,
a fourth expression cassette encoding the second heavy chain,
a fifth expression cassette encoding the second light chain,
a sixth expression cassette encoding the second light chain,
for the expression of the trivalent antibody in a mammalian cell.

4. A recombinant mammalian cell comprising a deoxyribonucleic acid encoding a trivalent antibody integrated in the genome of the cell, wherein the deoxyribonucleic acid encoding the trivalent antibody comprises in 5′- to 3′-direction
either (1)
a first expression cassette encoding the first heavy chain,
a second expression cassette encoding the first light chain,
a third expression cassette encoding the first light chain,
a fourth expression cassette encoding the second heavy chain, and
a fifth expression cassette encoding the second light chain,
or (2)
a first expression cassette encoding the first heavy chain,
a second expression cassette encoding the first light chain,
a third expression cassette encoding the first light chain,
a fourth expression cassette encoding the second heavy chain,
a fifth expression cassette encoding the second light chain,
a sixth expression cassette encoding the second light chain,
or (3)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second light chain,
or (4)
a first expression cassette encoding the first heavy chain,
a second expression cassette encoding the second heavy chain,
a third expression cassette encoding the first light chain,
a fourth expression cassette encoding the second light chain,
a fifth expression cassette encoding the first heavy chain,
a sixth expression cassette encoding the second light chain,
or (5)
a first expression cassette encoding the first heavy chain,
a second expression cassette encoding the second heavy chain,
a third expression cassette encoding the first light chain,
a fourth expression cassette encoding the second light chain,
a fifth expression cassette encoding the second heavy chain,
a sixth expression cassette encoding the second light chain,
a seventh expression cassette encoding the second light chain,
or (6)
a first expression cassette encoding the first light chain,
a second expression cassette encoding the first light chain,
a third expression cassette encoding the first heavy chain,
a fourth expression cassette encoding the second heavy chain,
a fifth expression cassette encoding the second light chain,
a sixth expression cassette encoding the second light chain.

5. A composition comprising two deoxyribonucleic acids, which comprise in turn three different recombination recognition sequences and five to seven expression cassettes, wherein
the first deoxyribonucleic acid comprises in 5′- to 3′-direction,
either (1)
a first recombination recognition sequence,
a first expression cassette encoding the first heavy chain,
a second expression cassette encoding the first light chain,
a third expression cassette encoding the first light chain, and
a first copy of a third recombination recognition sequence,
or (2)
a first recombination recognition sequence,
a first expression cassette encoding the first heavy chain,
a second expression cassette encoding the first light chain,
a third expression cassette encoding the first light chain, and
a first copy of a third recombination recognition sequence,
or (3)
a first recombination recognition sequence,
a first expression cassette encoding the first heavy chain,
a second expression cassette encoding the second heavy chain,
a third expression cassette encoding the first light chain,
a fourth expression cassette encoding the second light chain, and
a first copy of a third recombination recognition sequence,
or (4)
a first recombination recognition sequence,
a first expression cassette encoding the first heavy chain,
a second expression cassette encoding the second heavy chain,
a third expression cassette encoding the first light chain,
a fourth expression cassette encoding the second light chain, and
a first copy of a third recombination recognition sequence,
or (5)
a first recombination recognition sequence,
a first expression cassette encoding the first heavy chain,
a second expression cassette encoding the second heavy chain,
a third expression cassette encoding the first light chain,
a fourth expression cassette encoding the second light chain, and
a first copy of a third recombination recognition sequence,
or (6)
a first recombination recognition sequence,
a first expression cassette encoding the first light chain,
a second expression cassette encoding the first light chain,
a third expression cassette encoding the first heavy chain, and
a first copy of a third recombination recognition sequence,
and
the second deoxyribonucleic acid comprises in 5′- to 3′-direction
either (1)
a second copy of the third recombination recognition sequence,
a fourth expression cassette encoding the second heavy chain,
a fifth expression cassette encoding the second light chain, and
a second recombination recognition sequence,
or (2)
a second copy of the third recombination recognition sequence,
a fourth expression cassette encoding the second heavy chain,
a fifth expression cassette encoding the second light chain,
a sixth expression cassette encoding the second light chain, and
a second recombination recognition sequence,
or (3)
a second copy of the third recombination recognition sequence,
a fifth expression cassette encoding the second light chain, and
a second recombination recognition sequence,
or (4)
a second copy of the third recombination recognition sequence,
a fifth expression cassette encoding the first heavy chain,
a sixth expression cassette encoding the second light chain, and
a second recombination recognition sequence,
or (5)
a second copy of the third recombination recognition sequence,
a fifth expression cassette encoding the second heavy chain,
a sixth expression cassette encoding the second light chain,
a seventh expression cassette encoding the second light chain, and
a second recombination recognition sequence,
or (6)
a second copy of the third recombination recognition sequence,
a fourth expression cassette encoding the second heavy chain,
a fifth expression cassette encoding the second light chain,
a sixth expression cassette encoding the second light chain, and
a second recombination recognition sequence.

6. A method for producing a recombinant mammalian cell comprising a deoxyribonucleic acid encoding a trivalent antibody and secreting the trivalent antibody, comprising the following steps:
a) providing a mammalian cell comprising an exogenous nucleotide sequence integrated at a single site within a locus of the genome of the mammalian cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different;
b) introducing into the cell provided in a) a composition of two deoxyribonucleic acids comprising three different recombination recognition sequences and five to seven expression cassettes, wherein
the first deoxyribonucleic acid comprises in 5′- to 3′-direction,
either (1)
a first recombination recognition sequence,
a first expression cassette encoding the first heavy chain,
a second expression cassette encoding the first light chain,
a third expression cassette encoding the first light chain, and
a first copy of a third recombination recognition sequence,
or (2)
a first recombination recognition sequence,
a first expression cassette encoding the first heavy chain,
a second expression cassette encoding the first light chain,
a third expression cassette encoding the first light chain, and
a first copy of a third recombination recognition sequence,
or (3)
a first recombination recognition sequence,
a first expression cassette encoding the first heavy chain,
a second expression cassette encoding the second heavy chain,
a third expression cassette encoding the first light chain,
a fourth expression cassette encoding the second light chain, and
a first copy of a third recombination recognition sequence,
or (4)
a first recombination recognition sequence,
a first expression cassette encoding the first heavy chain,
a second expression cassette encoding the second heavy chain,
a third expression cassette encoding the first light chain,
a fourth expression cassette encoding the second light chain, and
a first copy of a third recombination recognition sequence,
or (5)
a first recombination recognition sequence,
a first expression cassette encoding the first heavy chain,
a second expression cassette encoding the second heavy chain,
a third expression cassette encoding the first light chain,
a fourth expression cassette encoding the second light chain, and
a first copy of a third recombination recognition sequence,
or (6)
a first recombination recognition sequence,
a first expression cassette encoding the first light chain,
a second expression cassette encoding the first light chain,
a third expression cassette encoding the first heavy chain, and
a first copy of a third recombination recognition sequence,
and
the second deoxyribonucleic acid comprises in 5′- to 3′-direction
either (1)
a second copy of the third recombination recognition sequence,
a fourth expression cassette encoding the second heavy chain,
a fifth expression cassette encoding the second light chain, and
a second recombination recognition sequence,
or (2)
a second copy of the third recombination recognition sequence,
a fourth expression cassette encoding the second heavy chain,
a fifth expression cassette encoding the second light chain,
a sixth expression cassette encoding the second light chain, and
a second recombination recognition sequence,
or (3)
a second copy of the third recombination recognition sequence,
a fifth expression cassette encoding the second light chain, and
a second recombination recognition sequence,
or (4)
a second copy of the third recombination recognition sequence,
a fifth expression cassette encoding the first heavy chain,
a sixth expression cassette encoding the second light chain, and
a second recombination recognition sequence,
or (5)
a second copy of the third recombination recognition sequence,
a fifth expression cassette encoding the second heavy chain,
a sixth expression cassette encoding the second light chain,
a seventh expression cassette encoding the second light chain, and
a second recombination recognition sequence,
or (6)
a second copy of the third recombination recognition sequence,
a fourth expression cassette encoding the second heavy chain,
a fifth expression cassette encoding the second light chain,
a sixth expression cassette encoding the second light chain, and
a second recombination recognition sequence,
wherein the first to third recombination recognition sequences of the first and second deoxyribonucleic acids are matching the first to third recombination recognition sequence on the integrated exogenous nucleotide sequence,
wherein the 5′-terminal part and the 3′-terminal part of the expression cassette encoding the one second selection marker when taken together form a functional expression cassette of the one second selection marker;
c) introducing
i) either simultaneously with the first and second deoxyribonucleic acid of b);
or
ii) sequentially thereafter
one or more recombinases,
wherein the one or more recombinases recognize the recombination recognition sequences of the first and the second deoxyribonucleic acid; (and optionally wherein the one or more recombinases perform two recombinase mediated cassette exchanges)
and
d) selecting for cells expressing the second selection marker and secreting the trivalent antibody,
thereby producing a recombinant mammalian cell comprising a deoxyribonucleic acid encoding the trivalent antibody and secreting the trivalent antibody.

7. The method for producing a trivalent antibody or the deoxyribonucleic acid or the use or the recombinant mammalian cell or the composition or the method for producing a recombinant mammalian cell according to claim 1, wherein the first heavy chain comprises in the CH3 domain the mutation T366W (numbering according to Kabat) and the second heavy chain comprises in the CH3 domain the mutations T366S, L368A, and Y407V (numbering according to Kabat), or vice versa.

8. The method for producing a trivalent antibody or the deoxyribonucleic acid or the use or the recombinant mammalian cell or the composition or the method for producing a recombinant mammalian cell according to claim 7, wherein one of the heavy chains further comprises the mutation S354C and the respective other heavy chain comprises the mutation Y349C.

9. The method for producing a trivalent antibody or the deoxyribonucleic acid or the use or the recombinant mammalian cell or the composition or the method for producing a recombinant mammalian cell according to claim 1, wherein the first heavy chain is an extended heavy chain comprising an additional domain exchanged Fab fragment.

10. The method for producing a trivalent antibody or the deoxyribonucleic acid or the use or the recombinant mammalian cell or the composition or the method for producing a recombinant mammalian cell according to claim 1, wherein the first light chain is a domain exchanged light chain VH-VL or CH1-CL.

11. The method for producing a trivalent antibody or the deoxyribonucleic acid or the use or the recombinant mammalian cell or the composition or the method for producing a recombinant mammalian cell according to claim 1, wherein
the first heavy chain comprises from N- to C-terminus a first heavy chain variable domain, a CH1 domain, a first light chain variable domain, a CH1 domain, a hinge region, a CH2 domain and a CH3 domain,
the second heavy chain comprises from N- to C-terminus the first heavy chain variable domain, a CH1 domain, a hinge region, a CH2 domain and a CH3 domain,
the first light chain comprises from N- to C-terminus a second heavy chain variable domain and a CL domain, and
the second light chain comprises from N- to C-terminus a second light chain variable domain and a CL domain,
wherein the first heavy chain variable domain and the second light chain variable domain form a first binding site and the second heavy chain variable domain and the first light chain variable domain form a second binding site.

12. The method for producing a trivalent antibody or the deoxyribonucleic acid or the use or the recombinant mammalian cell or the composition or the method for producing a recombinant mammalian cell according to claim 1, wherein the deoxyribonucleic acid is stably integrated into the genome of the mammalian cell at a single site or locus.

13. The method for producing a trivalent antibody or the deoxyribonucleic acid or the use or the recombinant mammalian cell or the composition or the method for producing a recombinant mammalian cell according to claim 1, wherein the deoxyribonucleic acid encoding the trivalent antibody comprises a further expression cassette encoding for a selection marker, wherein the expression cassette encoding for the selection marker is located partly 5′ and partly 3′ to the third recombination recognition sequence, wherein the 5′-located part of said expression cassette comprises the promoter and the start-codon and the 3′-located part of said expression cassette comprises the coding sequence without a start-codon and a polyA signal, wherein the start-codon is operably linked to the coding sequence.

14. The method for producing a trivalent antibody or the deoxyribonucleic acid or the use or the recombinant mammalian cell or the composition or the method for producing a recombinant mammalian cell according to claim 1, wherein the promoter is the human CMV promoter with intron A, the polyadenylation signal sequence is the bGH polyadenylation signal sequence and the terminator is the hGT terminator except for the expression cassette of the selection marker, wherein the promoter is the SV40 promoter and the polyadenylation signal sequence is the SV40 polyadenylation signal sequence and a terminator is absent.

15. The method for producing a trivalent antibody or the deoxyribonucleic acid or the use or the recombinant mammalian cell or the composition or the method for producing a recombinant mammalian cell according to claim 1, wherein the mammalian cell is a CHO cell.

16. The method for producing a trivalent antibody or the deoxyribonucleic acid or the use or the recombinant mammalian cell or the composition or the method for producing a recombinant mammalian cell according to claim 1, wherein when the organization in 5′ to 3′-direction has as the first expression cassette, an expression cassette encoding the first heavy chain, all cassettes are arranged unidirectional.

17. The method for producing a trivalent antibody or the deoxyribonucleic acid or the use or the recombinant mammalian cell or the composition or the method for producing a recombinant mammalian cell according to claim 1, wherein when the organization in the 5′ to 3′-direction is first expression cassette encoding the first light chain, second expression cassette encoding the first light chain, third expression cassette encoding the first heavy chain, fourth expression cassette encoding the second heavy chain, fifth expression cassette encoding the second light chain, sixth expression cassette encoding the second light chain, the first to third expression cassettes are arranged unidirectional and the fourth to sixth expression cassette are arranged unidirectional, whereby the first to third expression cassettes are arranged in opposite direction of the fourth to sixth expression cassette.


SEQUENCE LISTING (11 sequences)

SEQ ID NO: 1 - DNA, 34 bp, Artificial Sequence
  L3
ataacttcgtataaagtctcctatacgaagttat

SEQ ID NO: 2 - DNA, 34 bp, Artificial Sequence
  2L
ataacttcgtatagcatacattatacgaagttat

SEQ ID NO: 3 - DNA, 34 bp, Artificial Sequence
  loxFas
acaacttcgtatatacctttctatacgaagttgt

SEQ ID NO: 4 - DNA, 608 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 5 - DNA, 696 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 6 - DNA, 2125 bp, Human cytomegalovirus
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 7 - DNA, 129 bp, Simian virus 40
aacttgtttattgcagcttataatggttacaaataaagcaatagcatcacaaatttcacaaataaagcatttttttcaccattctagttgtggtttgtccaaactcatcaatgtatcttatcatgtctg

SEQ ID NO: 8 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 73 bp, Homo sapiens
caggataatatatggtagggttcatagccagagtaacctttttttttaatttttattttattttatttttgag

SEQ ID NO: 10 - DNA, 288 bp, Simian virus 40
agtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 11 - DNA, 798 bp, Artificial Sequence
  green fluorescent protein encoding nucleic acid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagatga